SEQUENCE LISTING (8 sequences)

SEQ ID NO: 1 - protein, 1991 aa, synthetic construct
RQFVKEKLLKWAESPAALTEVTVADTEPTRTQTLLQSLGELVKGVYAKYWIYVCAGMFIVVSFAGRLVVYKIVYMFLFLLCLTLFQVYYSLWRKLLKAFWWLVVAYTMLVLIAVYTFQFQDFPAYWRNLTGFTDEQLGDLGLEQFSVSELFSSILVPGFFLLACILQLHYFHRPFMQLTDMEHVSLPGTRLPRWAHRQDAVSGTPLLREEQQEHQQQQQEEEEEEEDSRDEGLGVATPHQATQVPEGAAKWGLVAERLLELAAGFSDVLSRVQVFLRRLLELHVFKLVALYTVWVALKEVSVMNLLLVVLWAFALPYPRFRPMASCLSTVWTCVIIVCKMLYQLKVVNPQEYSSNCTEPFPNSTNLLPTEISQSLLYRGPVDPANWFGVRKGFPNLGYIQNHLQVLLLLVFEAIVYRRQEHYRRQHQLAPLPAQAVFASGTRQQLDQDLLGCLKYFINFFFYKFGLEICFLMAVNVIGQRMNFLVTLHGCWLVAILTRRHRQAIARLWPNYCLFLALFLLYQYLLCLGMPPALCIDYPWRWSRAVPMNSALIKWLYLPDFFRAPNSTNLISDFLLLLCASQQWQVFSAERTEEWQRMAGVNTDRLEPLRGEPNPVPNFIHCRSYLDMLKVAVFRYLFWLVLVVVFVTGATRISIFGLGYLLACFYLLLFGTALLQRDTRARLVLWDCLILYNVTVIISKNMLSLLACVFVEQMQTGFCWVIQLFSLVCTVKGYYDPKEMMDRDQDCLLPVEEAGIIWDSVCFFFLLLQRRVFLSHYYLHVRADLQATALLASRGFALYNAANLKSIDFHRRIEEKSLAQLKRQMERIRAKQEKHRQGRVDRSRPQDTLGPKDPGLEPGPDSPGGSSPPRRQWWRPWLDHATVIHSGDYFLFESDSEEEEEAVPEDPRPSAQSAFQLAYQAWVTNAQAVLRRRQQEQEQARQEQAGQLPTGGGPSQEVEPAEGPEEAAAGRSHVVQRVLSTAQFLWMLGQALVDELTRWLQEFTRHHGTMSDVLRAERYLLTQELLQGGEVHRGVLDQLYTSQAEATLPGPTEAPNAPSTVSSGLGAEEPLSSMTDDMGSPLSTGYHTRSGSEEAVTDPGEREAGASLYQGLMRTASELLLDRRLRIPELEEAELFAEGQGRALRLLRAVYQCVAAHSELLCYFIIILNHMVTASAGSLVLPVLVFLWAMLSIPRPSKRFWMTAIVFTEIAVVVKYLFQFGFFPWNSHVVLRRYENKPYFPPRILGLEKTDGYIKYDLVQLMALFFHRSQLLCYGLWDHEEDSPSKEHDKSGEEEQGAEEGPGVPAATTEDHIQVEARVGPTDGTPEPQVELRPRDTRRISLRFRRRKKEGPARKGAAAIEAEDREEEEGEEEKEAPTGREKRPSRSGGRVRAAGRRLQGFCLSLAQGTYRPLRRFFHDILHTKYRAATDVYALMFLADVVDFIIIIFGFWAFGKHSAATDITSSLSDDQVPEAFLVMLLIQFSTMVVDRALYLRKTVLGKLAFQVALVLAIHLWMFFILPAVTERMFNQNVVAQLWYFVKCIYFALSAYQIRCGYPTRILGNFLTKKYNHLNLFLFQGFRLVPFLVELRAVMDWVWTDTTLSLSSWMCVEDIYANIFIIKCSRETEKKYPQPKGQKKKKIVKYGMGGLIILFLIAIIWFPLLFMSLVRSVVGVVNQPIDVTVTLKLGGYEPLFTMSAQQPSIIPFTAQAYEELSRQFDPQPLAMQFISQYSPEDIVTAQIEGSSGALWRISPPSRAQMKRELYNGTADITLRFTWNFQRDLAKGGTVEYANEKHMLALAPNSTARRQLASLLEGTSDQSVVIPNLFPKYIRAPNGPEANPVKQLQPNEEADYLGVRIQLRREQGAGATGFLEWWVIELQECRTDCNLLPMVIFSDKVSPPSLGFLAGYGIMGLYVSIVLVIGKFVRGFFSEISHSIMFEELPCVDRILKLCQDIFLVRETRELELEEELYAKLIFLYRSPETMIKWTREKE

SEQ ID NO: 2 - protein, 6 aa, synthetic construct
LPCALL

SEQ ID NO: 3 - protein, 234 aa, synthetic construct
RSVVGVVNQPIDVTVTLKLGGYEPLFTMSAQQPSIIPFTAQAYEELSRQFDPQPLAMQFISQYSPEDIVTAQIEGSSGALWRISPPSRAQMKRELYNGTADITLRFTWNFQRDLAKGGTVEYANEKHMLALAPNSTARRQLASLLEGTSDQSVVIPNLFPKYIRAPNGPEANPVKQLQPNEEADYLGVRIQLRREQGAGATGFLEWWVIELQECRTDCNLLPMVIFSDKVSPPS

SEQ ID NO: 4 - DNA, 7566 bp, synthetic construct
  source 1..7566: mol_type = other DNA
atggagccgcacgtgctcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatattgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgactttcaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtag

SEQ ID NO: 5 - DNA, 18 bp, synthetic construct
  source 1..18: mol_type = other DNA
ctgccctgcgcgctgctg

SEQ ID NO: 6 - DNA, 237 bp, synthetic construct
  source 1..237: mol_type = other DNA
cagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagc

SEQ ID NO: 7 - protein, 19 aa, synthetic construct
ATNFSLLKQAGDVEENPGP

SEQ ID NO: 8 - DNA, 8140 bp, synthetic construct
  source 1..8140: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctatggagccgcacgtgctcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatattgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgactttcaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtagggcagcggcgccacaaacttctctctgctaaagcaagcaggtgatgttgaagaaaaccccgggcct

(19) United States
(12) Patent Application Publication
(10) Pub. No.: US 2026/0049278 A1
(43) Pub. Date: Feb. 19, 2026

(54) MECHANO-GENETIC ENGINEERING OF PIEZO1-ENHANCED CAR T-CELLS FOR EFFICIENT IMMUNOTHERAPY

(71) Applicant: New York University, New York, NY (US)

(72) Inventors: Weiqiang Chen, New York, NY (US); Ngoc Luu, Brooklyn, NY (US)

(21) Appl. No.: 19/300,903

(22) Filed: Aug. 15, 2025

Related U.S. Application Data
(60) Provisional application No. 63/684,051, filed on Aug. 16, 2024.

(51) Int. Cl. C12N 5/0783; C07K 14/705; C07K 14/725; C07K 16/28

(52) U.S. Cl. CPC C12N 5/0636; C07K 14/705; C07K 14/7051; C07K 14/70521; C07K 14/70578; C07K 16/2803; C07K 2317/53; C07K 2317/622; C07K 2319/03; C12N 2510/00

(57) ABSTRACT

The invention provides compositions comprising immune cells modified to express a chimeric antigen receptor (CAR) and a mechanosensitive ligand, and methods of producing the same. The invention also provides methods of treating, preventing, or diagnosing cancer in a subject comprising administering to the subject an immune cell modified to express a CAR and a mechanosensitive ligand.

### CROSS-REFERENCE TO RELATED APPLICATIONS

This application claims priority to U.S. Provisional Application No. 63/684,051 filed on Aug. 16, 2024, incorporated herein by reference in its entirety.

### FEDERALLY SPONSORED RESEARCH AND DEVELOPMENT

This invention was made with government support under R35 GM133646 awarded by the National Institutes of Health. The government has certain rights in the invention.

### REFERENCE TO SEQUENCE LISTING SUBMITTED ELECTRONICALLY

This application contains a Sequence Listing, which is submitted electronically via EFS-Web as an XML Document formatted sequence listing with a file name “206256-0106-00US_SequenceListing” creation date of Aug. 11, 2025, and having a size of 26,217 bytes. The sequence listing submitted via EFS-Web is part of the specification and is herein incorporated by reference in its entirety.

### BACKGROUND OF THE INVENTION

Chimeric antigen receptor (CAR) T-cell therapy has become a paradigm-shifting therapeutic strategy in pilot clinical trials targeting different tumors, especially for B-cell malignancies (June et al., Science, 2018. 359(6382): p. 1361-1365; Labanieh et al., Nature Biomedical Engineering, 2018. 2(6): p. 377). However, the clinical efficacy of CAR T-cell therapy varies tremendously across trials, and overall patient responses remain unfavorable (Porter et al., Science translational medicine, 2015. 7(303): p. 303-139). This variability arises from multiple factors, including the potency and functional patterns of costimulatory domains incorporated into CAR designs, the intrinsic qualities of patients' T-cells, the efficiency of CAR T-cell manufacturing, and the in vivo resistance processes triggered by antigen escape, T-cell exhaustion, and immunosuppression within the tumor microenvironment (Li et al., Advanced Science, 2020. 7(24): p. 2002628; Chang et al., Nature chemical biology, 2018. 14(3): p. 317).

Thus, there is a need to develop novel techniques to improve CAR T-cell function, thereby improving patients' responses. The present invention addresses this need.

### SUMMARY OF THE INVENTION

In some embodiments, the present invention relates to a method of producing a Chimeric Antigen Receptor (CAR)-expressing immune cell comprising introducing a first nucleic acid sequence encoding a CAR and a second nucleic acid sequence encoding a mechanosensitive ligand to an immune cell.

In some embodiments, the immune cell is selected from T cells, NK cells, and macrophages. In some embodiments, the immune cell is a T cell.

In some embodiments, the first nucleic acid sequence encodes a CAR that binds to a tumor-specific antigen.

In some embodiments, the first nucleic acid sequence encodes a CAR that binds CD19, BCMA, CD123, or Mesothelin.

In some embodiments, the first nucleic acid sequence encodes a CAR molecule comprising a CD8 leader sequence, a CD19-scFv, a CD28-hinge region, a transmembrane domain derived from CD28 and/or 4-1BB, and a cytoplasmic signaling domain of CD3ζ.

In some embodiments, the second nucleic acid sequence encodes a mechanosensitive ligand selected from the group consisting of Piezo1, a fragment thereof, and a variant thereof. In some embodiments, the second nucleic acid sequence encodes the ΔL15-20 extracellular loop and/or F2114 residue from Piezo1.

In some embodiments, the second nucleic acid sequence encodes an amino acid sequence comprising SEQ ID NO:1. In some embodiments, the second nucleic acid sequence encodes an amino acid sequence comprising SEQ ID NO:2 and/or SEQ ID NO:3.

In some embodiments, the method comprises contacting the immune cell with a first nucleic acid molecule comprising the first sequence, and a second nucleic acid molecule comprising the second sequence.

In some embodiments, the method comprises contacting the immune cell with a nucleic acid molecule comprising the first and second nucleic acid sequence.

In some embodiments, the second nucleic acid sequence comprises SEQ ID NO:4.

In some embodiments, the second nucleic acid sequence comprises SEQ ID NO:5 and/or SEQ ID NO:6.

In some embodiments, the nucleic acid molecule comprises a nucleic acid sequence encoding a fusion protein comprising a CAR and a mechanosensitive ligand linked by a self-cleaving peptide. In some embodiments, the self-cleaving peptide comprises P2A. In some embodiments, the nucleic acid molecule comprises a nucleic acid sequence encoding SEQ ID NO:7.

In some embodiments, the invention relates to an immune cell genetically engineered to express: (a) a Chimeric Antigen Receptor (CAR); and (b) a mechanosensitive ligand.

In some embodiments, the immune cell is a T cell, an NK cell, or a macrophage. In some embodiments, the immune cell is a T cell.

In some embodiments, the CAR binds to a tumor-specific antigen. In some embodiments, the CAR binds to an antigen selected from the group consisting of CD19, BCMA, CD123, or Mesothelin.

In some embodiments, the CAR comprises a CD8 leader sequence, a CD19-scFv, a CD28-hinge region, a transmembrane domain derived from CD28 and/or 4-1BB, and a cytoplasmic signaling domain of CD3ζ.

In some embodiments, the mechanosensitive ligand is selected from the group consisting of Piezo1, a fragment thereof, or a variant thereof.

In some embodiments, the mechanosensitive ligand comprises the ΔL15-20 extracellular loop and/or F2114 residue from Piezo1.

In some embodiments, the mechanosensitive ligand comprises the amino acid sequence of SEQ ID NO:1.

In some embodiments, the mechanosensitive ligand comprises the amino acid sequence of SEQ ID NO:2 and/or SEQ ID NO:3.

In some embodiments, the invention relates to a population of CAR-expressing immune cells comprising a plurality of immune cells genetically engineered to express: (a) a Chimeric Antigen Receptor (CAR); and (b) a mechanosensitive ligand.

In some embodiments, the invention relates to a composition comprising an immune cell genetically engineered to express: (a) a Chimeric Antigen Receptor (CAR); and (b) a mechanosensitive ligand, or a population of immune cells genetically engineered to express: (a) a Chimeric Antigen Receptor (CAR); and (b) a mechanosensitive ligand.

In some embodiments, the invention relates to a method of treating or preventing cancer in a subject comprising administering to the subject an effective amount of a composition comprising an immune cell genetically engineered to express: (a) a Chimeric Antigen Receptor (CAR); and (b) a mechanosensitive ligand, or a population of immune cells genetically engineered to express: (a) a Chimeric Antigen Receptor (CAR); and (b) a mechanosensitive ligand

In some embodiments, the cancer is selected from the group consisting of leukemia, pancreatic cancer, and breast cancer.

In some embodiments, the invention relates to a nucleic acid molecule comprising: (a) a first nucleic acid sequence encoding a CAR; and (b) a second nucleic acid sequence encoding a mechanosensitive ligand.

In some embodiments, the first nucleic acid sequence encodes a CAR that binds a tumor-specific antigen.

In some embodiments, the first nucleic acid sequence encodes a CAR that binds an antigen selected from the group consisting of CD19, BCMA, CD123, and Mesothelin.

In some embodiments, the first nucleic acid sequence encodes a CAR molecule comprising a CD8 leader sequence, a CD19-scFv, a CD28-hinge region, a transmembrane domain derived from CD28 and/or 4-1BB, and a cytoplasmic signaling domain of CD3ζ.

In some embodiments, the second nucleic acid sequence encodes a mechanosensitive ligand selected from the group consisting of Piezo1, a fragment thereof, or a variant thereof.

In some embodiments, the second nucleic acid sequence encodes the ΔL15-20 extracellular loop and/or F2114 residue from Piezo1.

In some embodiments, the second nucleic acid sequence encodes an amino acid sequence of SEQ ID NO:1.

In some embodiments, the second nucleic acid sequence encodes an amino acid sequence of SEQ ID NO:2 and/or SEQ ID NO:3.

In some embodiments, the second nucleic acid sequence comprises SEQ ID NO:4.

In some embodiments, the second nucleic acid sequence comprises SEQ ID NO:5 and/or SEQ ID NO:6.

In some embodiments, the nucleic acid molecule further comprises a nucleic acid sequence encoding a self-cleaving peptide. In some embodiments, the self-cleaving peptide comprises P2A. In some embodiments, the self-cleaving peptide comprises an amino acid sequence of SEQ ID NO:7.

In some embodiments, the nucleic acid molecule encodes a fusion protein comprising a CAR and a mechanosensitive ligand linked by a self-cleaving peptide.

In some embodiments, the nucleic acid molecule comprises a nucleic acid sequence of SEQ ID NO:8. In some embodiments, the nucleic acid molecule comprises a nucleic acid sequence of SEQ ID NO:8 linked to a nucleic acid sequence encoding a CAR.

In some embodiments, the invention relates to a composition comprising a first nucleic acid molecule and a second nucleic acid molecule, wherein the first nucleic acid molecule comprises a nucleic acid sequence encoding a CAR, and the second nucleic acid molecule comprises a nucleic acid sequence encoding a mechanosensitive ligand.

In some embodiments, the first nucleic acid molecule encodes a CAR that binds a tumor-specific antigen. In some embodiments, the first nucleic acid molecule encodes a CAR that binds an antigen selected from the group consisting of CD19, BCMA, CD123, and Mesothelin.

In some embodiments, the first nucleic acid molecule encodes a CAR molecule comprising a CD8 leader sequence, a CD19-scFv, a CD28-hinge region, a transmembrane domain derived from CD28 and/or 4-1BB, and a cytoplasmic signaling domain of CD3ζ.

In some embodiments, the second nucleic acid molecule encodes a mechanosensitive ligand selected from the group consisting of Piezo1, a fragment thereof, and a variant thereof.

In some embodiments, the second nucleic acid molecule encodes the ΔL15-20 extracellular loop and/or F2114 residue from Piezo1.

In some embodiments, the second nucleic acid molecule encodes an amino acid sequence comprising SEQ ID NO:1.

In some embodiments, the second nucleic acid molecule encodes an amino acid sequence comprising SEQ ID NO:2 and/or SEQ ID NO:3.

In some embodiments, the second nucleic acid molecule comprises a nucleic acid sequence of SEQ ID NO:4.

In some embodiments, the second nucleic acid molecule comprises a nucleic acid sequence of SEQ ID NO:5 and/or SEQ ID NO:6.

### BRIEF DESCRIPTION OF THE DRAWINGS

The following detailed description of exemplary embodiments of the invention will be better understood when read in conjunction with the appended drawings. For the purpose of illustrating the invention, there are shown in the drawings exemplary. It should be understood, however, that the invention is not limited to the precise arrangements and instrumentalities of the embodiments shown in the drawings.

FIG. 1A and FIG. 1B depict results of example experiments demonstrating synaptic force dynamics of CAR T-cells upon CD19 activation. FIG. 1A depicts PDMS micropillar array substrate functionalized with CD19, ICAM-1, and fibrinogen for force sensing. FIG. 1B depicts CAR T-cell synaptic force magnitude and direction dynamics.

FIG. 2A-FIG. 2G depict results of example experiments demonstrating Piezo1 signaling in CAR T-cell activation. FIG. 2A depicts RT-qPCR results of mechanotransductive signaling. FIG. 2B depicts Piezo1/Ca²⁺ signaling gene expression. FIG. 2C depicts immunofluorescence (IF) images of Piezo1 expression 1 hour alter activation and intracellular calcium in CAR T-cells. FIG. 2D depicts quantification of Piezo1 expression in FIG. 2C, and FIG. 2E depicts quantification of intracellular calcium in FIG. 2C. FIG. 2F depicts IF images of Piezo1 intensity in activated and inactivated T-cells, and FIG. 2G depicts the quantification of FIG. 2F. Scale bar=5 μm.

FIG. 3A-FIG. 3D depict results of example experiments demonstrating actomyosin activities during immunological synapse (IS) formation. FIG. 3A depicts staining images of actin and myosin IIA in CAR T-cells. FIG. 3B depicts the spreading area of activated and nonactivated CAR T-cells. FIG. 3C depicts the actin distribution in activated and nonactivated CAR T-cells. FIG. 3D depicts the quantification of maximum actin velocity, characteristic frequency, and amplitude of actin dynamics with frequency spectra analysis.

FIG. 4A-FIG. 4D depict results of example experiments exploring the cytoskeleton machinery underlying CAR T-cell activation with Rac1 and RhoA FRET biosensors. FIG. 4A depicts the overall dynamics of Rac1 activities during CAR T-cell activation. FIG. 4B depicts ratiometric images of Rac1 activities during CAR T-cell activation. FIG. 4C depicts correlation of synaptic force and Rac1 activities in CAR T-cells. FIG. 4D depicts the spatial and temporal dynamics of RhoA activities in cells in 2D and 3D culture measured by RhoA FRET sensor.

FIG. 5A-FIG. 5C depict results of example experiments demonstrating that genetic modification of Piezo1 influences T-cell activation and mechanotransduction. FIG. 5A depicts a schematic of example experiments to genetically modulate Piezo1 expression in CAR T-cells using Piezo1-specific CRISPR plasmids for gene knockout and activation of Piezo1. FIG. 5B depicts staining of Piezo1 in Piezo1-modified T-cells. FIG. 5C depicts the expression of activation markers CD69 and CD25 in Piezo1-modified T-cells.

FIG. 6A-FIG. 6E depict results of example experiments demonstrating the mechano-phenotype of patient-derived CAR T-cells. FIG. 6A depicts a schematic of Piezo1 expression in healthy and patient-derived CAR T-cells. FIG. 6B depicts the expression levels of exhaustion makers PD-1 and LAG-3, and immune functional cytokines IFNγ, IL-2, and TNFα in CAR T-cells between healthy and patient-derived CAR T-cells. FIG. 6C depicts a frequency spectrum in healthy and patient-derived CAR T-cells. FIG. 6D depicts Rac1 dynamics in healthy and patient-derived CAR T-cells. FIG. 6E depicts Piezo1 expression in healthy and patient-derived CAR T-cells.

FIG. 7A-FIG. 7C depict results of example experiments of CAR T-cell synaptic force measurement. FIG. 7A depicts PDMS micropillar array force sensor showing CAR T-cell exhibits outward pushing force on micropillars in the spreading stage, while the CAR centralization stage requires inward contractile force. Scale bar=5 μm. FIG. 7B depicts force dynamics of healthy and patient-derived CAR T-cells. FIG. 7C depicts instantaneous frequency of healthy and patient-derived CAR T-cells.

FIG. 8A-FIG. 8E depict results of example experiments demonstrating the mechano-phenotype of exhausted CAR T-cells. FIG. 8A depicts a schematic of experiments to induce T-cell exhaustion. FIG. 8B depicts the expression levels of exhaustion makers PD-1 and LAG-3, and immune functional cytokines IFNγ, IL-2, and TNFα in CAR T-cells upon CD19 activation. FIG. 8C depicts instantaneous frequency spectra in healthy and exhausted CAR T-cells. FIG. 8D depicts Rac1 dynamics in healthy and exhausted CAR T-cells. FIG. 8E depicts Piezo1 expression in healthy and exhausted CAR T-cells.

FIG. 9A-FIG. 9C depict the engineering of mechano-enhanced Piezo1-CAR T-cell via co-transduction. FIG. 9A depicts the Piezo1 overexpression lentiviral construct.

FIG. 9B depicts the standard CD19 4-1BBζ-CAR construct containing an antigen recognition domain (scFv), CD28 hinge, transmembrane domain CD28, 4-1BB costimulatory domain, and CD3ζ activation domain. FIG. 9C depicts the manufacturing of a Piezo1-CAR T-cell.

FIG. 10B-FIG. 10C depict the engineering of mechano-enhanced Piezo1-CAR T-cell using CAR-PIEZO1 bicistronic vector. FIG. 10A depicts the bicistronic vector construct, containing two open reading frames separated by a 2A “self-cleaving” peptide sequence P2A. FIG. 10B depicts the manufacturing of the Piezo1-CAR T-cell. FIG. 10C depicts the function of the Piezo1-CAR T-cell.

FIG. 11 depicts the human leukemia-on-a-chip model for CAR T-cell therapy screening.

FIG. 12A-FIG. 12D depict the results of example experiments monitoring the human CAR T-cell dynamics in a leukemia-on-a-chip model. FIG. 12A depicts extravasation of CAR T-cells in the bone marrow niche. FIG. 12B depicts infiltration of CAR T-cells in the bone marrow niche. FIG. 12C depicts recognition and killing activity of CAR T-cells in the bone marrow niche. FIG. 12D depicts increased various cytokines in the niche upon CAR T-cell therapy.

### DETAILED DESCRIPTION

The present invention relates to immune cells modified to express a chimeric antigen receptor (CAR) and a mechanosensitive ligand, or variants or fragments thereof. In some embodiments, the present invention relates to methods of producing immune cells modified to express a CAR and a mechanosensitive ligand, or variants or fragments thereof.

In some embodiments, the method comprises introducing to an immune cell a first nucleic acid molecule comprising a nucleic acid sequence encoding a CAR, or a variant or fragment thereof, and a second nucleic acid molecule comprising a nucleic acid sequence encoding a mechanosensitive ligand, or a variant or fragment thereof. In some embodiments, the method comprises introducing to an immune cell a nucleic acid molecule comprising a nucleic acid sequence encoding a CAR, or a variant or fragment thereof, and a nucleic acid sequence encoding a mechanosensitive ligand, or a variant or fragment thereof.

In some embodiments, the invention relates to nucleic acid molecules comprising a nucleic acid sequence encoding a CAR, or a variant or fragment thereof, and nucleic acid molecules comprising a nucleic acid sequence encoding a mechanosensitive ligand, or a variant or fragment thereof. In some embodiments, the invention relates to nucleic acid molecules comprising a first nucleic acid sequence encoding a CAR, or a variant or fragment thereof, and a second nucleic acid sequence encoding a mechanosensitive ligand, or a variant or fragment thereof.

In some embodiments, the present invention relates to methods of treating cancer or a disease or disorder associated therewith in a subject in need thereof, comprising administering to the subject an immune cell expressing a CAR and a mechanosensitive ligand, or variants or fragments thereof.

### Definitions

Unless defined otherwise, all technical and scientific terms used herein have the same meaning as commonly understood by one of ordinary skill in the art to which the invention pertains.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

As used herein, the term “Chimeric Antigen Receptor” or alternatively a “CAR” refers to a recombinant polypeptide construct comprising at least an extracellular antigen binding domain, a transmembrane domain, and a cytoplasmic signaling domain comprising a functional signaling domain derived from a stimulatory molecule as defined below. In one aspect, the stimulatory molecule is the zeta chain associated with the T cell receptor complex. In one aspect, the cytoplasmic signaling domain further comprises one or more functional signaling domains derived from at least one costimulatory molecule as defined below. In one aspect, the costimulatory molecule is chosen from 4-1BB (i.e., CD137), CD3, and/or CD28. In one aspect, the CAR comprises a chimeric fusion protein comprising an extracellular antigen recognition domain, a transmembrane domain, and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the scFv domain during cellular processing and localization of the CAR to the cellular membrane.

The portion of the CAR composition comprising an antibody or antibody fragment thereof may exist in a variety of forms where the antigen binding domain is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In one embodiment, the CAR comprises an antibody fragment that comprises a scFv.

As used herein, a “signaling domain” is the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies are typically tetramers of immunoglobulin molecules.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)₂, and Fv fragments, scFv antibody fragments, linear antibodies, single domain antibodies such as sdAb (either VL or VH), camelid VHH domains, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

By the term “recombinant antibody” as used herein, is meant an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a cell or a fluid with other biological components.

As used herein, the term “immune cell” includes cells that are of hematopoietic origin and that play a role in the immune response. Immune cells include lymphocytes, such as B cells and T cells; natural killer cells; myeloid cells, such as monocytes, macrophages, dendritic cells, eosinophils, mast cells, basophils, and granulocytes.

As used herein, the term “immune response” includes T cell mediated and/or B cell mediated immune responses that are influenced by modulation of T cell co-stimulation. The term immune response further includes immune responses that are indirectly affected by T cell activation such as antibody production (humoral responses) and the activation of cytokine responsive cells such as macrophages.

As used herein, the term “T cell immune response” refers to activation of antigen specific T cells as measured by proliferation or expression of molecules on the cell surface or secretion of proteins such as cytokines.

As used herein, the term “T cell” refers to a lymphocyte (e.g., white blood cell) that functions in cell-mediated immunity. In some embodiments, the presence of a T cell receptor (TCR) on the cell surface distinguishes T cells from other lymphocytes. As is known in the art, T cells typically do not present antigens, and rely on other lymphocytes (e.g., natural killer cells and B cells) to aid in antigen presentation. Types of T cells include: T helper cells (TH cells), Memory T cells (Tcm, Tem, or Temra), Regulatory T cells (Treg), Cytotoxic T cells (CTLs), Natural killer T cells (NK cells), gamma delta T cells, and Mucosal associated invariant T cells (MAIT).

As used herein, the term “TCR” refers to “T cell receptor.” A T cell receptor is a molecule on the surface of T lymphocytes (“T cells”). In embodiments, the receptor is an ap-TCR receptor, meaning that the T cell receptor comprises an alpha (a) and beta (p) chain, which is typically expressed as part of a complex with CD3 chain molecules.

As used herein, the term “B cell” refers to a cell produced in the bone marrow of an animal expressing membrane-bound antibody specific for an antigen. Following interaction with the antigen it differentiates into a plasma cell producing antibodies specific for the antigen or into a memory B cell. “B cell” and “B lymphocyte” are used interchangeably. Naïve as well as activated B cells are within the scope of the invention.

As used herein, the term “antigen-specific B cell” refers to a B cell which expresses antibodies that are able to distinguish between the antigen of interest and other antigens and which specifically bind to that antigen of interest with high or low affinity but which do not bind to other antigens.

As used herein, the term “positive B cell” means any B cell which is labeled with any one of the labeling compounds of the invention and which is selected or sorted or otherwise separated from a mixture of cells by a device capable of detecting said labeling compound. For example, a B cell which is positive for the first labeling compound of the invention is a B cell which is labeled with a first labeling compound and which is selected by the device capable of detecting said first labeling compound.

As used herein, the term “B cell marker” refers to surface molecules on the B cells which are specific for antigen-specific IgG-producing B cells. B cell markers suitable for use in the present invention include, but are not limited to surface IgG, kappa and lambda chains, Ig-alpha (CD79alpha), Ig-beta (CD79beta), CD19, Ia, Fc receptors, B220 (CD45R), CD20, CD21, CD22, CD23, CD81 (TAPA-1) or any other CD antigen specific for B cells.

As used herein, the term “autologous” is meant to refer to any material derived from the same individual to whom it is later to be re-introduced into the individual.

As used herein, the term “conservative sequence modifications” is intended to refer to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody or antibody fragment of the invention can be replaced with other amino acid residues from the same side chain family and the altered antibody or antibody fragment can be tested for the ability to bind TCR using the functional assays described herein.

An “antigen presenting cell” or “APC” as used herein, means an immune system cell such as an accessory cell (e.g., a B-cell, a dendritic cell, and the like) that displays foreign antigens complexed with HLA I, HLA II, MHC I, or MHC II complexes on their surfaces. For example, T cells may recognize these complexes using their T-cell receptors (TCRs). APCs process antigens and present them to T-cells.

As used herein “zeta” or alternatively “zeta chain”, “CD3-zeta” or “TCR-zeta” is defined as the protein provided as GenBan acc. no. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect, the cytoplasmic domain of zeta comprises residues 52 through 164 of GenBank acc. no. BAG36664.1 or the equivalent residues from a non-human species, e.g., mouse, rodent, monkey, ape and the like, that are functional orthologs thereof.

A “costimulatory molecule” refers to the cognate binding partner on a cell that specifically binds with a costimulatory ligand, thereby mediating a costimulatory response by the cell, such as, but not limited to, proliferation. Costimulatory molecules are cell surface molecules other than antigen receptors or their ligands that are required for an efficient immune response. Costimulatory molecules include, but are not limited to an HLA class I molecule, HLA class II molecule, MHC class I molecule, MHC class II molecule, MHC class III molecule, BTLA and a Toll ligand receptor, as well as OX40, CD3, CD27, CD28, CDS, ICAM-1, LFA-1 (CD11a/CD18) and 4-1BB (CD137).

As used herein “4-1BB” is defined as member of the TNFR superfamily with an amino acid sequence provided as GenBank accno. AAA62478.2, or the equivalent residues from a non-human species, e.g., mouse, rodent, monkey, ape and the like; and a “4-1BB costimulatory domain” are defined amino acid residues 214-255 of GenBank accno. AAA62478.2, or the equivalent residues from a non-human species, e.g., mouse, rodent, monkey, ape and the like. In one aspect, the “4-1BB costimulatory domain” is a sequence derived from a non-human species, e.g., mouse, rodent, monkey, ape and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of at least one sign or symptom of the disease or disorder, the frequency with which such a sign or symptom is experienced by a patient, or both, is reduced.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to further include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “retrovirus” refers to a retroviral particle which is capable of entering a cell (i.e., the particle contains a membrane-associated protein such as an envelope protein or a viral G glycoprotein which can bind to the host cell surface and facilitate entry of the viral particle into the cytoplasm of the host cell) and integrating the retroviral genome (as a double-stranded provirus) into the genome of the host cell. The term “retrovirus” encompasses Oncovirinae (e.g., Moloney murine leukemia virus (MoMLV), Moloney murine sarcoma virus (MoMSN), and Mouse mammary tumor virus (MMTN), Spumavirinae, and Lentivirinae (e.g., Hum.an immunodeficiency virus, Simian immunodeficiency virus, Equine infection anemia virus, and Caprine arthritis-encephalitis virus; See, e.g., U.S. Pat. Nos. 5,994,136 and 6,013,516, both of which are incorporated herein by reference).

As used herein, the term “retroviral vector” refers to a retrovirus that has been modified to express a gene of interest. Retroviral vectors can be used to transfer genes efficiently into host cells by exploiting the viral infectious process. Foreign or heterologous genes cloned (i.e., inserted using molecular biological techniques) into the retroviral genome can be delivered efficiently to host cells that are susceptible to infection by the retrovirus. Through well-known genetic manipulations, the replicative capacity of the retroviral genome can be destroyed. The resulting replication-defective vectors can be used to introduce new genetic material to a cell but they are unable to replicate. A helper virus or packaging cell line can be used to permit vector particle assembly and egress from the cell. Such retroviral vectors comprise a replication-deficient retroviral genome containing a nucleic acid sequence encoding at least one gene of interest (i.e., a polycistronic nucleic acid sequence can encode more than one gene of interest), a 5′ retroviral long terminal repeat (5′ LTR); and a 3′ retroviral long terminal repeat (3′ LTR). The term “pseudotyped retroviral vector” refers to a retroviral vector containing a heterologous membrane protein.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

A “lentiviral vector” is a vector derived from at least a portion of a lentivirus genome, including especially a self-inactivating lentiviral vector as provided in Milone et al., Mol. Ther. 17(8): 1453-1464 (2009). Other Examples or lentivirus vectors that may be used in the clinic as an alternative to the pELPS vector, include but not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies/antibody fragments can comprise residues which are found neither in the recipient antibody nor in the imported CDR or framework sequences. These modifications are made to further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, where necessary to join two protein coding regions, are in the same reading frame.

“Parenteral” administration of an immunogenic composition includes, e.g., subcutaneous (s.c.), intravenous (i.v.), intramuscular (i.m.), or intrasternal injection, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, recombinant peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “flexible polypeptide linker” refers to a peptide linker that consists of amino acids such as glycine and serine residues used alone or in combination, to link variable heavy and variable light chain regions together. In one embodiment, the flexible polypeptide linker is a Gly/Ser linker and comprises the amino acid sequence (Gly-Gly-Gly-Ser)n, where n is a positive integer equal to or greater than 1. For example, n=1, n=2, n=3. n=4, n=5 and n=6, n=7, n=8, n=9 and n=10. In one embodiment, the flexible polypeptide linkers include, but are not limited to, (Gly4 Ser)4 or (Gly4 Ser)3 In another embodiment, the linkers include multiple repeats of (Gly2Ser), (GlySer) or (Gly3Ser). Also included within the scope of the invention are linkers described in WO2012/138475, incorporated herein by reference).

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human, mouse).

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In some embodiments, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

By the term “specifically binds,” as used herein, is meant an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Provided herein are immune cells modified to express a chimeric antigen receptor (CAR) and a mechanosensitive ligand, or variants or fragments thereof. In some embodiments, the present invention relates to methods of producing immune cells modified to express a CAR and a mechanosensitive ligand, or variants or fragments thereof.

In some embodiments, the method comprises introducing to an immune cell a first nucleic acid molecule comprising a nucleic acid sequence encoding a CAR, or a variant or fragment thereof, and a second nucleic acid molecule comprising a nucleic acid sequence encoding a mechanosensitive ligand, or a variant or fragment thereof. In some embodiments, the method comprises introducing to an immune cell a nucleic acid molecule comprising a nucleic acid sequence encoding a CAR, or a variant or fragment thereof, and a nucleic acid sequence encoding a mechanosensitive ligand, or a variant or fragment thereof.

Further provided herein are nucleic acid molecules comprising a nucleic acid sequence encoding a CAR, or a variant or fragment thereof, and nucleic acid molecules comprising a nucleic acid sequence encoding a mechanosensitive ligand, or a variant or fragment thereof. In some embodiments, the invention relates to nucleic acid molecules comprising a first nucleic acid sequence encoding a CAR, or a variant or fragment thereof, and a second nucleic acid sequence encoding a mechanosensitive ligand, or a variant or fragment thereof.

In some embodiments, provided herein are methods of administering to a subject an immune cell expressing a CAR and a mechanosensitive ligand, or variants or fragments thereof. In some embodiments, provided herein are methods of treating cancer or a disease or disorder associated therewith in a subject in need thereof, comprising administering to the subject an immune cell expressing a CAR and a mechanosensitive ligand, or variants or fragments thereof.

### Compositions

In some embodiments, the present invention relates to immune cells modified to express a chimeric antigen receptor (CAR) and a mechanosensitive ligand, or variants or fragments thereof. In some embodiments, the invention provides a nucleic acid molecule comprising a nucleic acid sequence encoding a CAR. In some embodiments, the invention provides a nucleic acid molecule comprising a nucleic acid sequence encoding a mechanosensitive ligand. In some embodiments, the invention provides a nucleic acid molecule comprising a nucleic acid sequence encoding a CAR and a nucleic acid sequence encoding a mechanosensitive ligand.

### Chimeric Antigen Receptor (CAR)

In some embodiments, the CAR comprises a means for binding an antigen. In some embodiments, the CAR comprises an antigen binding domain. In some embodiments, the antigen binding domain is a targeting domain, wherein the targeting domain directs the cell expressing the CAR to a cell or particle expressing a target receptor.

In various embodiments, the CAR can be any CAR molecule including, but not limited to, a “first generation,” “second generation,” “third generation,” “fourth generation” or “fifth generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the invention comprise an antigen binding domain, for example, a single-chain variable fragment (scFv), fused to a transmembrane domain, which is fused to a cytoplasmic/intracellular domain of a T cell receptor chain. “First generation” CARs typically have the intracellular domain from the CD3ζ-chain, which is the primary transmitter of signals from endogenous T cell receptors (TCRs). “First generation” CARs can provide de novo antigen recognition and cause activation of both CD4+ and CD8+ T cells through their CD3ζ chain signaling domain in a single fusion molecule, independent of HLA-mediated antigen presentation.

“Second-generation” CARs for use in the invention comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)).

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD3ζ signaling domain in addition to a constitutive or inducible chemokine component.

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2Rβ.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

A CAR can be generated using well known methods for designing a CAR, including those as described herein. A CAR, whether a first, second, third, fourth or fifth generation CAR, can be readily designed by fusing an antigen binding domain, to an immune cell signaling domain, such as a T cell receptor cytoplasmic/intracellular domain. As described above, the CAR generally has the structure of a cell surface receptor, with the antigen binding activity, such as an scFv, as at least a portion of the extracellular domain, fused to a transmembrane domain, which is fused to an intracellular domain that has cell signaling activity in a T cell. The CAR can include co-stimulatory molecules, as described herein. One skilled in the art can readily select appropriate transmembrane domains, as described herein and known in the art, and intracellular domains to provide the desired signaling capability in the T cell.

In some embodiments, the CAR comprises a means for binding a tumor antigen. In some embodiments, the antigen binding domain of the CAR binds a tumor antigen. In some embodiments, the antigen binding domain binds to CD19, CD20, CD22, CD38, CD123, CD138, BCMA, Mesothelin, CD33, or PTK7. Therefore, in some embodiments, the CAR comprises a means for binding a tumor antigen selected from CD19, CD20, CD22, CD38, CD123, CD138, BCMA, Mesothelin, CD33, or PTK7. In some embodiments, the antigen binding domain binds to CD19.

In one embodiment, the antigen binding domain of the CAR of the invention comprises an antibody or fragment thereof. The antibody can be expressed as an immunoglobulin, for example, an IgG, or as a Bi-specific T-cell engager (BiTE), a diabody, a duel affinity re-targeting antibody (DART), a Fab, a F(ab′), a single chain variable fragment (scFv), a nanobody, a bi-specific antibody, or the like.

In some embodiments, the antigen binding domain can be an scFv or a Fab, or any suitable antigen binding fragment of an antibody (see Sadelain et al., Cancer Discov. 3:38-398 (2013)). Many antibodies or antigen binding domains derived from antibodies that bind to an antigen, such as a cancer antigen, are known in the art. Alternatively, such antibodies or antigen binding domains can be produced by routine methods. Methods of generating an antibody are well known in the art, including methods of producing a monoclonal antibody or screening a library to obtain an antigen binding polypeptide, including screening a library of human Fabs (Winter and Harris, Immunol. Today 14:243-246 (1993); Ward et al., Nature 341:544-546 (1989); Harlow and Lane, Antibodies: A Laboratory Manual, Cold Spring Harbor Laboratory Press (1988); Hilyard et al., Protein Engineering: A practical approach (IRL Press 1992); Borrabeck, Antibody Engineering, 2nd ed. (Oxford University Press 1995); Huse et al., Science 246:1275-1281 (1989)). For the CAR, the antigen binding domain derived from an antibody can be human, humanized, chimeric, CDR-grafted, and the like, as desired. For example, if a mouse monoclonal antibody is a source antibody for generating the antigen binding domain of a CAR, such an antibody can be humanized by grafting CDRs of the mouse antibody onto a human framework (see Borrabeck, supra, 1995), which can be beneficial for administering the CAR to a human subject. In a preferred embodiment, the antigen binding domain is an scFv. The generation of scFvs is well known in the art (see, for example, Huston, et al., Proc. Nat. Acad. Sci. USA 85:5879-5883 (1988); Ahmad et al., Clin. Dev. Immunol. 2012: ID980250 (2012); U.S. Pat. Nos. 5,091,513, 5,132,405 and 4,956,778; and U.S. Patent Publication Nos. 20050196754 and 20050196754)).

In some embodiments, a CAR can comprise a signaling domain derived from a CD3ζ polypeptide, for example, a signaling domain derived from the intracellular domain of CD3ζ, which can activate or stimulate an immune cell. CD3ζ comprises 3 Immune-receptor-Tyrosine-based-Activation-Motifs (ITAMs), and transmits an activation signal to the cell, for example, a cell of the lymphoid lineage, such as a T cell, after antigen is bound. It is understood that a “CD3ζ nucleic acid molecule” refers to a polynucleotide encoding a CD3ζ polypeptide.

In certain non-limiting embodiments, an intracellular domain of a CAR can further comprise at least one co-stimulatory signaling domain. Such a co-stimulatory signaling domain can provide increased activation of an immune cell. A co-stimulatory signaling domain can be derived from a CD28 polypeptide, a 4-1BB polypeptide, an OX40 polypeptide, an ICOS polypeptide, a DAP10 polypeptide, a 2B4 polypeptide, and the like. In some embodiments, the intracellular domain of a CAR can comprise a co-stimulatory signaling region that comprises two co-stimulatory molecules, such as CD28 and 4-1BB, or other combinations of co-stimulatory ligands, as disclosed herein.

In some embodiments, the antigen binding domain of a CAR can be fused to a leader or a signal peptide that directs the nascent protein into the endoplasmic reticulum and subsequent translocation to the cell surface. It is understood that, once a polypeptide containing a signal peptide is expressed at the cell surface, the signal peptide has generally been proteolytically removed during processing of the polypeptide in the endoplasmic reticulum and translocation to the cell surface. Thus, in some embodiments, a polypeptide such as a CAR is expressed at the cell surface as a mature protein lacking the signal peptide, whereas the precursor form of the polypeptide includes the signal peptide. The signal sequence or leader is a peptide sequence generally present at the N-terminus of newly synthesized proteins that directs their entry into the secretory pathway. The signal peptide is covalently joined to the N-terminus of the extracellular antigen-binding domain of a CAR as a fusion protein. Any suitable signal peptide, as are well known in the art, can be applied to a CAR to provide cell surface expression in an immune cell (see Gierasch Biochem. 28:923-930 (1989); von Heijne, J. Mol. Biol. 184 (1):99-105 (1985)). Exemplary signal peptides can be derived from cell surface proteins naturally expressed in an immune cell, including any of the signal peptides of the polypeptides disclosed herein. Thus, any suitable signal peptide can be utilized to direct a CAR to be expressed at the cell surface of an immune cell.

In certain non-limiting embodiments, an antigen-binding domain of a CAR can comprise a linker sequence or peptide linker connecting the heavy chain variable region and light chain variable region of the antigen-binding domain. In certain non-limiting embodiments, a CAR can also comprise a spacer region or sequence that links the domains of the CAR to each other. For example, a spacer can be included between a signal peptide and an antigen binding domain, between the antigen binding domain and the transmembrane domain, between the transmembrane domain and the intracellular domain, and/or between domains within the intracellular domain, for example, between a stimulatory domain and a co-stimulatory domain. The spacer region can be flexible enough to allow interactions of various domains with other polypeptides, for example, to allow the antigen binding domain to have flexibility in orientation in order to facilitate antigen recognition. The spacer region can be, for example, the hinge region from an IgG, the CH2CH3 (constant) region of an immunoglobulin, and/or portions of CD3 (cluster of differentiation 3) or some other sequence suitable as a spacer.

In some embodiments, the transmembrane domain of a CAR comprises a hydrophobic alpha helix that spans at least a portion of the membrane. Different transmembrane domains result in different receptor stability. After antigen recognition, receptors cluster and a signal is transmitted to the cell. In an embodiment, the transmembrane domain of a CAR can be derived from another polypeptide that is naturally expressed in the immune cell. In one embodiment, a CAR can have a transmembrane domain derived from CD8, CD28, CD3, CD4, 4-1BB, OX40, ICOS, CTLA-4, PD-1, LAG-3, 2B4, BTLA, or other polypeptides expressed in the immune cell having a transmembrane domain, including others as disclosed herein or that are well known in the art. In some embodiments, the CAR has a transmembrane domain derived from CD28 and/or 4-1BB. Optionally, the transmembrane domain can be derived from a polypeptide that is not naturally expressed in the immune cell, so long as the transmembrane domain can function in transducing signal from antigen bound to the CAR to the intracellular signaling and/or co-stimulatory domains. It is understood that the portion of the polypeptide that comprises a transmembrane domain of the polypeptide can include additional sequences from the polypeptide, for example, additional sequences adjacent on the N-terminal or C-terminal end of the transmembrane domain, or other regions of the polypeptide, as desired.

It is understood that domains of the polypeptides described herein can be used in a CAR, as useful to provide a desired function such as a signal peptide, antigen binding domain, transmembrane domain, intracellular signaling domain and/or co-stimulatory domain. For example, a domain can be selected such as a signal peptide, a transmembrane domain, an intracellular signaling domain, or other domain, as desired, to provide a particular function to a CAR of the invention. Possible desirable functions can include, but are not limited to, providing a signal peptide and/or transmembrane domain.

Exemplary CARs include those described, for example, in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282. Exemplary CARs also include any described in Marofi et al., Stem Cell Res Ther 12: 81 (2021); Townsend et al., J Exp Clin Cancer Res 37: 163 (2018); Ma et al., Int J Biol Sci 15(12): 2548-2560 (2019); Zhao and Cao, Front Immunol 10: 2250 (2019); Han et al., J Cancer 12(2): 326-334 (2021); Specht et al., Cancer Res 79: 4 Supplement, Abstract P2-09-13; Byers et al., Journal of Clinical Oncology 37, no. 15_suppl (2019); Panowski et al., Cancer Res 79 (13 Supplement) 2326 (2019); and Sauer et al., Blood 134 (Supplement_1): 1932 (2019); or can contain any of the antibodies or antigen-binding fragments described in U.S. Pat. Nos. 8,153,765; 8,603,477, 8,008,450; U.S. Pub. No. US20120189622 or US20100260748; and International PCT Publication Nos. WO2006099875, WO2009080829, WO2012092612, WO2014210064.

Further exemplary CARs include the CARs of idecabtagene vicleucel, ABECMA®, BCMA02, JCARH125, JNJ-68284528 (LCAR-B38M; ciltacabtagene autoleucel; CARVYKTI™) (Janssen/Legend), P-BCMA-101 (Poseida), PBCAR269A (Poseida), P-BCMA-Allol (Poseida), Allo-715 (Pfizer/Allogene), CT053 (Carsgen), Descartes-08 (Cartesian), PHE885 (Novartis), ARI-002 (Hospital Clinic Barcelona, ID IB APS), and CTX120 (CRISPR Therapeutics). In a particular embodiment, the CAR is the CAR of idecabtagene vicleucel cells. In a particular embodiment, the CAR is the CAR of ABECMA® cells (cells used in ABECMA® immunotherapy). In a particular embodiment, the CAR is the CAR of ciltacabtagene autoleucel cells. In a particular embodiment, the CAR is the CAR of CARVYKTI™ cells (cells used in CARVYKTI™ immunotherapy).

Exemplary CARs also include the CARs of FDA-approved products BREYANZI® (lisocabtagene maraleucel), TECARTUS™ (brexucabtagene autoleucel), KYMRIAH™ (tisagenlecleucel), and YESCARTA™ (axicabtagene ciloleucel), ABECMA® (idecabtagene vicleucel), and CARVYKTI™ (ciltacabtagene autoleucel). In some of any of the provided embodiments, the CAR is the CAR of BREYANZI® (lisocabtagene maraleucel), TECARTUS™ (brexucabtagene autoleucel), KYMRIAH™ (tisagenlecleucel), YESCARTA™ (axicabtagene ciloleucel), ABECMA® (idecabtagene vicleucel), or CARVYKTI™ (ciltacabtagene autoleucel). In some of any of the provided embodiments, the CAR is the CAR of BREYANZI® (lisocabtagene maraleucel, see Sehgal et al., 2020, Journal of Clinical Oncology 38:15_suppl, 8040; Teoh et al., 2019, Blood 134(Supplement_1):593; and Abramson et al., 2020, The Lancet 396(10254): 839-852). In some of any of the provided embodiments, the CAR is the CAR of TECARTUS™ (brexucabtagene autoleucel, see Mian and Hill, 2021, Expert Opin Biol Ther; 21(4):435-441; and Wang et al., 2021, Blood 138(Supplement 1):744). In some of any of the provided embodiments, the CAR is the CAR of KYMRIAH™ (tisagenlecleucel, see Bishop et al., 2022, N Engl J Med 386:629:639; Schuster et al., 2019, N Engl J Med 380:45-56; Halford et al., 2021, Ann Pharmacother 55(4):466-479; Mueller et al., 2021, Blood Adv. 5(23):4980-4991; and Fowler et al., 2022, Nature Medicine 28:325-332). In some of any of the provided embodiments, the CAR is the CAR of YESCARTA™ (axicabtagene ciloleucel, see Neelapu et al., 2017, N Engl J Med 377(26):2531-2544; Jacobson et al., 2021, The Lancet 23(1):P91-103; and Locke et al., 2022, N Engl J Med 386:640-654). In some of any of the provided embodiments, the CAR is the CAR of ABECMA® (idecabtagene vicleucel, see Raje et al., 2019, N Engl J Med 380:1726-1737; and Munshi et al., 2021, N Engl J Med 384:705-716). In some of any of the provided embodiments, the CAR is the CAR of CARVYKTI™ (ciltacabtagene autoleucel, see Berdeja et al., Lancet. 2021 Jul. 24; 398(10297):314-324; and Martin, Abstract #549 [Oral], presented at 2021 American Society of Hematology (ASH) Annual Meeting & Exposition)).

In some embodiments, the CAR molecule comprises a CD8 leader sequence, a CD19-scFv, a CD28-hinge region, a transmembrane domain derived from CD28 and/or 4-1BB, and a cytoplasmic signaling domain of CD3ζ.

### Mechanosensitive Ligand

In various embodiments, the mechanosensitive ligand can be any ligand that affects the mechanical forces generated by the interaction between a CAR molecule and its corresponding antigen the surface of a cell.

In some embodiments, the mechanosensitive ligand is selected from the group consisting of Piezo1, or a variant or fragment thereof.

In some embodiments, the mechanosensitive ligand comprises Piezo1, a fragment thereof, or a variant thereof. In some embodiments, the mechanosensitive ligand comprises SEQ ID NO:1. In some embodiments, the mechanosensitive ligand comprises a fragment of Piezo1 comprising the ΔL15-20 extracellular loop. In some embodiments, the mechanosensitive ligand comprises SEQ ID NO:2. In some embodiments, the mechanosensitive ligand comprises a fragment of Piezo1 comprising the F2114 residue. In some embodiments, the mechanosensitive ligand comprises SEQ ID NO:3. In some embodiments, the mechanosensitive ligand comprises the ΔL15-20 extracellular loop and the F2114 residue from Piezo1. In some embodiments, the mechanosensitive ligand comprises SEQ ID NO:2 and SEQ ID NO:3.

In some embodiments, a variant of the mechanosensitive ligand as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over the full length of the amino acid sequence of SEQ ID NO: 1, SEQ ID NO:2, and/or SEQ ID NO:3.

In some embodiments, a fragment of the mechanosensitive ligand as described herein comprises at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full length amino acid sequence of SEQ ID NO:1, SEQ ID NO:2, and/or SEQ ID NO:3.

### Nucleic Acid Molecules

In some embodiments, the invention comprises a nucleic acid molecule comprising a nucleic acid sequence encoding a CAR. In various embodiments, the nucleic acid sequence can encode any CAR molecule including, but not limited to, a “first generation,” “second generation,” “third generation,” “fourth generation” or “fifth generation” CAR as described herein. In some embodiments, the nucleic acid sequence encoding the CAR encodes an antigen binding domain that targets a tumor-specific antigen as described herein. In some embodiments, the nucleic acid sequence encoding the CAR encodes a CD8 leader sequence, a CD19-scFv, a CD28-hinge region, a transmembrane domain derived from CD28 and/or 4-1BB, and a cytoplasmic signaling domain of CD3ζ, or a fragment or variant thereof.

In various embodiments, the invention comprises a nucleic acid molecule comprising a nucleic acid sequence encoding a mechanosensitive ligand described above. In some embodiments, the nucleic acid molecule comprises a nucleic acid sequence encoding a mechanosensitive ligand selected from the group consisting of Piezo1, a fragment thereof, or a variant thereof. In some embodiments, the nucleic acid molecule comprises a nucleic acid sequence encoding Piezo1. In some embodiments, the nucleic acid molecule comprises the nucleic acid sequence of SEQ ID NO:4, or a fragment or variant thereof. In some embodiments, the nucleic acid molecule comprises a nucleic acid sequence encoding the ΔL15-20 extracellular loop from Piezo1. In some embodiments, the nucleic acid molecule comprises the nucleic acid sequence of SEQ ID NO:5, or a fragment or variant thereof. In some embodiments, the nucleic acid molecule comprises a nucleic acid sequence encoding a fragment of Piezo1 comprising the F2114 residue. In some embodiments, the nucleic acid molecule comprises the nucleic acid sequence of SEQ ID NO:6, or a fragment or variant thereof. In some embodiments, the nucleic acid molecule comprises a nucleic acid sequence encoding the ΔL15-20 extracellular loop and F2114 residue from Piezo1. In some embodiments, the nucleic acid molecule comprises the nucleic acid sequence of SEQ ID NO:5 and SEQ ID NO:6, or fragments or variants thereof.

In some embodiments, the invention provides a nucleic acid molecule comprising a first sequence encoding a CAR and a second sequence encoding a mechanosensitive ligand. In various embodiments, the nucleic acid sequence can encode any CAR molecule including those described above. In some embodiments, the nucleic acid sequence encoding the CAR encodes an antigen binding domain that targets a tumor-specific antigen as described above. In some embodiments, the nucleic acid sequence encoding the CAR encodes a CD8 leader sequence, a CD19-scFv, a CD28-hinge region, a transmembrane domain derived from CD28 and/or 4-1BB, and a cytoplasmic signaling domain of CD3ζ, or a fragment or variant thereof.

In some embodiments, the nucleic acid molecule comprises a nucleic acid sequence encoding a mechanosensitive ligand selected from the group consisting of Piezo1, a fragment thereof, or a variant thereof. In some embodiments, the nucleic acid molecule comprises a nucleic acid sequence encoding Piezo1. In some embodiments, the nucleic acid molecule comprises the nucleic acid sequence of SEQ ID NO:4, or a fragment or variant thereof. In some embodiments, the nucleic acid molecule comprises a nucleic acid sequence encoding the ΔL15-20 extracellular loop from Piezo1. In some embodiments, the nucleic acid molecule comprises the nucleic acid sequence of SEQ ID NO:5, or a fragment or variant thereof. In some embodiments, the nucleic acid molecule comprises a nucleic acid sequence encoding a fragment of Piezo1 comprising the F2114 residue. In some embodiments, the nucleic acid molecule comprises the nucleic acid sequence of SEQ ID NO:6, or a fragment or variant thereof. In some embodiments, the nucleic acid molecule comprises a nucleic acid sequence encoding the ΔL15-20 extracellular loop and F2114 residue from Piezo1. In some embodiments, the nucleic acid molecule comprises the nucleic acid sequence of SEQ ID NO:5 and SEQ ID NO:6, or fragments or variants thereof.

In some embodiments, a variant of a nucleotide sequence encoding the mechanosensitive ligand as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over the full length of a nucleotide sequence of SEQ ID NO:4, SEQ ID NO:5, and/or SEQ ID NO:6.

In some embodiments, a fragment of a nucleotide sequence encoding the mechanosensitive ligand as described herein comprises at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full length nucleotide sequence of SEQ ID NO:4, SEQ ID NO:5, and/or SEQ ID NO:6.

In some embodiments, the nucleic acid molecule comprises a first nucleic acid sequence encoding a CAR molecule and a second nucleic acid sequence comprising SEQ ID NO:4, or a fragment or variant thereof. In some embodiments, the nucleic acid molecule comprises a first nucleic acid sequence encoding a CAR molecule and a second nucleic acid sequence comprising SEQ ID NO:5, or a fragment or variant thereof. In some embodiments, the nucleic acid molecule comprises a first nucleic acid sequence encoding a CAR molecule and a second nucleic acid sequence comprising SEQ ID NO:6, or a fragment or variant thereof. In some embodiments, the nucleic acid molecule comprises a first nucleic acid sequence encoding a CAR molecule and a second nucleic acid sequence comprising SEQ ID NO:5 and SEQ ID NO:6, or fragments or variants thereof.

In some embodiments, the nucleic acid molecule comprising a nucleic acid sequence encoding a CAR molecule and a nucleic acid sequence encoding a mechanosensitive ligand further comprises a nucleic acid sequence encoding a self-cleaving peptide sequence. In some embodiments, the self-cleaving peptide sequence is a P2A peptide sequence. In some embodiments, the self-cleaving peptide sequence comprises SEQ ID NO: 7.

In some embodiments, the nucleic acid molecule comprises a nucleic acid sequence encoding a CAR comprising a CD8 leader sequence, a CD19-scFv, a CD28-hinge region, a transmembrane domain derived from CD28 and/or 4-1BB, and a cytoplasmic signaling domain of CD3ζ, a nucleic acid sequence encoding peptide comprising SEQ ID NO:7, and a nucleic acid sequence comprising SEQ ID NO:5 and SEQ ID NO:6.

In some embodiments, the nucleic acid molecule comprises SEQ ID NO:8, or a fragment or variant thereof. In some embodiments, the nucleic acid molecule comprises SEQ ID NO:8 linked to a nucleic acid sequence encoding a CAR.

### Vectors

The present invention also provides vectors in which a nucleic acid molecule of the invention is inserted. In some embodiments, the vector is a DNA, a RNA, a plasmid, a lentivirus vector, an adenoviral vector, or a retrovirus vector. Vectors derived from retroviruses, such as the lentivirus, are suitable tools to achieve long-term gene transfer since they allow long-term, stable integration of a transgene and its propagation in daughter cells. Lentiviral vectors have the added advantage over vectors derived from onco-retroviruses, such as murine leukemia viruses, in that they can transduce non-proliferating cells, such as hepatocytes. They also have the added advantage of low immunogenicity.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, the vector comprising the nucleic acid molecule of the invention is an adenoviral vector (A5/35). In some embodiments, the expression of nucleic acids encoding CARs can be accomplished using transposons, such as sleeping beauty, crisper, CAS9, and zinc finger nucleases. See, for example, June et al. 2009 Nature Reviews Immunology 9.10: 704-716, which is incorporated herein by reference.

In some embodiments, the vector comprises a promoter. In some embodiments. additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1α promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from transgenes cloned into a lentiviral vector. See, e.g., Milone et al., Mol. Ther. 17(8): 1453-1464 (2009).

In order to assess the expression of polypeptide or portions thereof, the expression vector to be introduced into a cell can also contain either a selectable marker gene or a reporter gene or both to facilitate identification and selection of expressing cells from the population of cells sought to be transfected or infected through viral vectors. In various embodiments, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles.

In the case where a non-viral delivery system is utilized, an exemplary delivery vehicle is a liposome. The use of lipid formulations is contemplated for the introduction of the nucleic acids into a host cell (in vitro, ex vivo or in vivo). In one embodiment, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL.). Stock solutions of lipids in chloroform or chloroform/methanol can be stored at about −200 C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### Cells

In various aspects, the invention also provides a cell modified to express the CAR and mechanosensitive ligand described herein. In various embodiments, the cell is an immune cell, T cell, B cell, natural blood cell, white blood cell, myeloid cell, plasma cell, or any combination thereof.

Prior to expansion and genetic modification, a source of cells (e.g. immune cell, T cell, B cell, natural killer cell, white blood cell, myeloid cell, plasma cell, etc.) may be obtained from a subject. Examples of subjects include humans, dogs, cats, mice, rats, and transgenic species thereof. The cells can be obtained from a number of sources, including peripheral blood mononuclear cells, bone marrow, lymph node tissue, cord blood, thymus tissue, tissue from a site of infection, ascites, pleural effusion, spleen tissue, and tumors. In various embodiments of the present invention, any number of T cell lines available in the art, may be used. In various embodiments of the present invention, T cells can be obtained from a unit of blood collected from a subject using any number of techniques known to the skilled artisan, such as Ficoll™ separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In one embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In some embodiments, the cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In one embodiment, the time period ranges from 30 minutes to 36 hours or longer and all integer values there between. In one embodiment, the time period is at least 1, 2, 3, 4, 5, or 6 hours. In yet another embodiment, the time period is 10 to 24 hours. In one embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In various embodiments, it may be desirable to perform the selection procedure and use the “unselected” cells in the activation and expansion process. “Unselected” cells can also be subjected to further rounds of selection.

Enrichment of a cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In various embodiments, it may be desirable to enrich for or positively select for regulatory cells which typically express CD4+, CD25+, CD62Lhi, GITR+, and FoxP3+. Alternatively, in various embodiments, T regulatory cells are depleted by anti-C25 conjugated beads or other similar method of selection.

For isolation of a desired population of cells by positive or negative selection, the concentration of cells and surface (e.g., particles such as beads) can be varied. In various embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In one embodiment, greater than 100 million cells/ml is used. In one embodiment, a concentration of cells of 10, 15, 20, 25, 30, 35, 40, 45, or 50 million cells/ml is used. In one embodiment, a concentration of cells from 75, 80, 85, 90, 95, or 100 million cells/ml is used. In some embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In some embodiments, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10e6/ml. In some embodiments, the concentration used can be from about 1×10⁵/ml to 1×10⁶/ml, and any integer value in between.

In some embodiments, the cells may be incubated on a rotator for varying lengths of time at varying speeds at either 2-10° C. or at room temperature.

The cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In various embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or disorders that would benefit from T cell therapy, such as those described herein. In one embodiment a blood sample or an apheresis is taken from a generally healthy subject. In various embodiments, a blood sample or an apheresis is taken from a generally healthy subject who is at risk of developing a disease, but who has not yet developed a disease, and the cells of interest are isolated and frozen for later use. In various embodiments, the T cells may be expanded, frozen, and used at a later time. In various embodiments, samples are collected from a patient shortly after diagnosis of a particular disease as described herein but prior to any treatments. In one embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993). In one embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In one embodiment, the cells are isolated prior to and can be frozen for later use for treatment following B-cell ablative therapy such as agents that react with CD20, e.g., Rituxan.

In some embodiments of the present invention, T cells are obtained from a patient directly following treatment that leaves the subject with functional T cells. In this regard, it has been observed that following certain treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in various embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In some embodiments, the cell of the present invention is an immune cell modified to express a CAR molecule, a fragment thereof, or a variant thereof, and a mechanosensitive ligand, a fragment thereof, or a variant thereof. In some embodiments, the cell comprises a CAR molecule comprising a tumor-antigen binding domain. In some embodiments, the cell comprises a CAR molecule comprising a CD19 binding domain. In some embodiments, the cell comprises a CAR molecule comprising a CD8 leader sequence, a CD19-scFv, a CD28-hinge region, a transmembrane domain derived from CD28 and/or 4-1BB, and a cytoplasmic signaling domain of CD3ζ. In some embodiments, the cell comprises a mechanosensitive ligand comprising Piezo1, a fragment thereof, or a variant thereof. In some embodiments, the mechanosensitive ligand comprises ΔL15-20 extracellular loop and/or F2114 residue from Piezo1. In some embodiments, the cell comprises SEQ ID NO:1, SEQ ID NO:2, and/or SEQ ID NO:3, a fragment thereof, or a variant thereof. In some embodiments, the cell is a T cell.

### Methods

In some embodiments, the present invention relates to methods of modifying an immune cell to express a CAR, a fragment thereof, or variant thereof, and a mechanosensitive ligand, a fragment thereof, or a variant thereof. In some embodiments, the CAR comprises a tumor-antigen binding domain. In some embodiments, the CAR comprises a CD19 binding domain. In some embodiments, the CAR molecule comprises a CD8 leader sequence, a CD19-scFv, a CD28-hinge region, a transmembrane domain derived from CD28 and/or 4-1BB, and a cytoplasmic signaling domain of CD3ζ. In some embodiments, the mechanosensitive ligand comprises Piezo1, a fragment thereof, or a variant thereof. In some embodiments, the mechanosensitive ligand comprises the ΔL15-20 extracellular loop and/or F2114 residue from Piezo1. In some embodiments, the cell comprises SEQ ID NO:1, SEQ ID NO:2, and/or SEQ ID NO:3, a fragment thereof, or a variant thereof.

In some embodiments, the method comprises introducing a first nucleic acid sequence encoding a CAR, as described above, and a second nucleic acid sequence encoding a mechanosensitive ligand, as described above, to an immune cell. In some embodiments, the nucleic acid sequence encoding the CAR encodes a CD8 leader sequence, a CD19-scFv, a CD28-hinge region, a transmembrane domain derived from CD28 and/or 4-1BB, and a cytoplasmic signaling domain of CD3ζ, or a fragment or variant thereof. In some embodiments, the nucleic acid sequence encoding the mechanosensitive ligand comprises SEQ ID NO:4, SEQ ID NO:5 and/or SEQ ID NO:6, a fragment thereof, or a variant thereof.

In some embodiments, the method comprises contacting an immune cell with a first nucleic acid molecule comprising the first sequence encoding the CAR, and a second nucleic acid molecule comprising the second sequence encoding the mechanosensitive ligand. In some embodiments, the first nucleic acid molecule encodes a CAR molecule comprising a CD8 leader sequence, a CD19-scFv, a CD28-hinge region, a transmembrane domain derived from CD28 and/or 4-1BB, and a cytoplasmic signaling domain of CD3ζ, or a fragment or variant thereof. In some embodiments, the second nucleic acid molecule comprises SEQ ID NO:4, SEQ ID NO:5, and/or SEQ ID NO:6, a fragment thereof, or a variant thereof. In some embodiments, the nucleic acid molecule encodes SEQ ID NO:1, SEQ ID NO:2, and/or SEQ ID NO:3.

In some embodiments, the method comprises contacting an immune cell with a nucleic acid molecule comprising the first sequence encoding the CAR and the second sequence encoding the mechanosensitive ligand. In some embodiments, the nucleic acid molecule encodes a CAR molecule comprising a CD8 leader sequence, a CD19-scFv, a CD28-hinge region, a transmembrane domain derived from CD28 and/or 4-1BB, and a cytoplasmic signaling domain of CD3ζ, or a fragment or variant thereof. In some embodiments, the nucleic acid molecule comprises SEQ ID NO:4, SEQ ID NO:5, and/or SEQ ID NO:6, a fragment thereof, or a variant thereof. In some embodiments, the nucleic acid molecule encodes SEQ ID NO:1, SEQ ID NO:2, and/or SEQ ID NO:3.

In some embodiments, the nucleic acid molecule comprising a nucleic acid sequence encoding a CAR molecule and a nucleic acid sequence encoding a mechanosensitive ligand further comprises a nucleic acid sequence encoding a self-cleaving peptide sequence. In some embodiments, the self-cleaving peptide sequence is a P2A peptide sequence. In some embodiments, the self-cleaving peptide sequence comprises SEQ ID NO: 7.

In some embodiments, the method comprises contacting an immune cell with a nucleic acid molecule comprising a nucleic acid sequence encoding a CAR comprising a CD8 leader sequence, a CD19-scFv, a CD28-hinge region, a transmembrane domain derived from CD28 and/or 4-1BB, and a cytoplasmic signaling domain of CD3ζ, a nucleic acid sequence encoding peptide comprising SEQ ID NO:7, and a nucleic acid sequence comprising SEQ ID NO:5 and SEQ ID NO:6.

In some embodiments, the method comprises contacting an immune cell with a nucleic acid molecule comprising SEQ ID NO:8, or a fragment or variant thereof. In some embodiments, the method comprises contacting an immune cell with a nucleic acid molecule comprising SEQ ID NO:8 linked to a nucleic acid sequence encoding a CAR.

### Therapeutic Application

In some embodiments, the invention relates to methods of administering a modified immune cell expressing a CAR and a mechanosensitive ligand to a subject. In some embodiments, the immune cell is a T cell. In some embodiments, the modified immune cell is administered to a subject to diagnose or treat cancer.

The following are non-limiting examples of cancers that can be diagnosed or treated by the disclosed methods and compositions: acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytoma/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocytoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

The invention also relates to methods of treating a subject with immunotherapy, wherein the subject is in need of such therapy. In some embodiments the immunotherapy promotes an immune response. In some embodiments, the subject being treated may have cancer or pre-cancer, and administration of the modified immune cells of the invention is to treat the cancer or prevent progression of the cancer. The immune cells may be targeted to the cancer by virtue of expressing a CAR. In some embodiments the CAR binds to CD19 expressed on a tumor cell and administration of the modified immune cells of the invention treats the cancer. In one embodiment, the modified immune cell is a T cell. The T cell can be CD8+, CD4+, a TSCM, a TCM, effector memory T cell, effector T cell, Th1 cell, Th2 cell, Th9 cell, Th17 cell, Th22 cell, Tfh (follicular helper) cell, or other T cell as disclosed herein.

It is understood that a method of treating cancer can include any effect that ameliorates a sign or symptom associated with cancer. Such signs or symptoms include, but are not limited to, reducing the number of cancer cells, reducing tumor burden, including inhibiting growth of a tumor, slowing the growth rate of a tumor, reducing the size of a tumor, reducing the number of tumors, eliminating a tumor, all of which can be measured using routine tumor imaging techniques well known in the art. Other signs or symptoms associated with cancer include, but are not limited to, fatigue, pain, weight loss, and other signs or symptoms associated with various cancers. Thus, administration of the cells of the invention can reduce the number of tumor cells, reduce tumor size, and/or eradicate the tumor in the subject. The tumor can be a blood cancer or a solid tumor. The methods of the invention can also provide for increased or lengthened survival of a subject having cancer. Additionally, methods of the invention can provide for an increased immune response in the subject, for example, an increased immune response against the cancer.

In some embodiments, the cancer can involve a solid tumor. Cancers to be treated using the cells of the invention comprise cancers typically responsive to immunotherapy. Exemplary types of cancers include, but are not limited to, adrenocortical carcinoma (ACC); bladder urothelial carcinoma (BLCA); B-cell acute lymphoblastic leukemia (B-ALL); breast invasive carcinoma (BRCA); cervical squamous cell carcinoma and endocervical adenocarcinoma (CESC); cholangio carcinoma (CHOL); chronic lymphocytic leukemia (CLL); colon adenocarcinoma (COAD); lymphoid neoplasm diffuse large B-cell lymphoma (DLBC); esophageal carcinoma (ESCA); glioblastoma multiforme (GBM); head and neck squamous cell carcinoma (HNSC); kidney chromophobe (KICH); kidney renal clear cell carcinoma (KIRC); kidney renal papillary cell carcinoma (KIRP); acute myeloid leukemia (LAML); brain lower grade glioma (LGG); liver hepatocellular carcinoma (LIHC); lung adenocarcinoma (LUAD); lung squamous cell carcinoma (LUSC); mesothelioma (MESO); multiple myeloma (MM); ovarian serous cystadenocarcinora (OV); pancreatic adenocarcinoma (PAAD); pheochromocytoma and paraganglioma (PCPG); pancreatic ductal adenocarcinoma (PDAC); prostate adenocarcinoma (PRAD); rectum adenocarcinoma (READ); sarcoma (SARC); skin cutaneous melanoma (SKCM); stomach adenocarcinoma (STAD); testicular germ cell tumors (TGCT); thyroid carcinoma (THCA); thymoma (THYM); uterine corpus endometrial carcinoma (UCEC); uterine carcinosarcoma (UCS); and uveal Melanoma (UVM).

For treatment, the amount administered is an amount effective for producing the desired effect. An effective amount or therapeutically effective amount is an amount sufficient to provide a beneficial or desired clinical result upon treatment. An effective amount can be provided in a single administration or a series of administrations (one or more doses). An effective amount can be provided in a bolus or by continuous perfusion. In terms of treatment, an effective amount is an amount that is sufficient to palliate, ameliorate, stabilize, reverse or slow the progression of the disease, or otherwise reduce the pathological consequences of the disease. The effective amount can be determined by the physician for a particular subject. Several factors are typically taken into account when determining an appropriate dosage to achieve an effective amount. These factors include age, sex and weight of the subject, the condition being treated, the severity of the condition and the form and effective concentration of the cells of the invention being administered.

The cells of the invention are generally administered as a dose based on cells per kilogram (cells/kg) of body weight. Generally the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to 10⁶, depending on the mode and location of administration. In general, in the case of systemic administration, a higher dose is used than in regional administration, where the immune cells of the invention are administered in the region, an organ or a tumor. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 1×10⁸, 6×10⁴ to 1×10⁸, 7×10⁴ to 1×10⁸, 8×10⁴ to 1×10⁸, 9×10⁴ to 1×10⁸, 1×10⁵ to 1×10⁸, and the like. Such dose ranges can be particularly useful for regional administration. In a particular embodiment, cells are provided in a dose of 1×10⁵ to 5×10⁶, in particular 1×10⁵ to 3×10⁶ or 3×10⁵ to 3×10⁶ cells/kg for regional administration, for example, intrapleural administration. The dose can also be adjusted to account for whether a single dose is being administered or whether multiple doses are being administered. The precise determination of what would be considered an effective dose can be based on factors individual to each subject, including their size, age, sex, weight, and condition of the particular subject, as described above. Dosages can be readily determined by those skilled in the art based on the disclosure herein and knowledge in the art.

The cells of the invention can be administered by any methods known in the art, including, but not limited to, pleural administration, intravenous administration, subcutaneous administration, intranodal administration, intratumoral administration, intrathecal administration, intrapleural administration, intraperitoneal administration, intracranial administration, and direct administration to the thymus. In one embodiment, the cells of the invention can be delivered regionally to an organ, a tumor or site of an autoimmune disease or site of an infectious disease using well known methods, including but not limited to, hepatic or aortic pump; limb, lung or liver perfusion; in the portal vein; through a venous shunt; in a cavity or in a vein that is nearby a tumor, and the like. In another embodiment, the cells of the invention can be administered systemically. In still another embodiment, the cells are administered regionally at the site of a desired therapy, for example, at the site of a tumor. In the case of a tumor, the cells can also be administered intratumorally, for example, by direct injection of the cells at the site of a tumor and/or into the tumor vasculature. One skilled in the art can select a suitable mode of administration based on the type of target tissue or target region and/or location of a target tissue or target region to be treated. The cells can be introduced by injection or catheter. Optionally, expansion and/or differentiation agents can be administered to the subject prior to, during or after administration of cells to increase production of the cells of the invention in vivo.

In some embodiments, proliferation of the cells of the invention is done ex vivo, prior to administration to a subject, or in vivo after administration to a subject (see Kaiser et al., Cancer Gene Therapy 22:72-78 (2015)).

The methods of the invention can further comprise adjuvant therapy in combination with, either prior to, during, or after treatment with the cells of the invention. Thus, the cell therapy methods of the invention can be used with other standard care and/or therapies that are compatible with administration of the cells of the invention.

### Pharmaceutical Compositions

Pharmaceutical compositions of the present invention may comprise a modified immune cell as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one embodiment formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount” or “therapeutic amount” is indicated, the precise amount of the compositions of the present invention to be administered can be determined by a physician with consideration of individual differences in age, weight, extent of infection or metastasis, and condition of the patient (subject). It can generally be stated that a pharmaceutical composition comprising the cells described herein may be administered at a dosage of 10e4 to 10e9 cells/kg body weight, in some instances 10e5 to 10e6 cells/kg body weight, including all integer values within those ranges. The cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In various embodiments, it may be desired to administer activated cells to a subject and then subsequently redraw blood (or have an apheresis performed), activate cells therefrom according to the present invention, and reinfuse the patient with these activated and expanded cells. This process can be carried out multiple times every few weeks. In various embodiments, cells can be activated from blood draws of from 10 cc to 400 cc. In various embodiments, cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol, may select out certain populations of cells.

The administration of the subject compositions may be carried out in any convenient manner, including by aerosol inhalation, injection, ingestion, transfusion, implantation or transplantation. The compositions described herein may be administered to a patient transarterially, subcutaneously, intradermally, intranodally, intramedullary, intramuscularly, by intravenous (i.v.) injection, or intraperitoneally. In one embodiment, the T cell compositions of the present invention are administered to a patient by intradermal or subcutaneous injection. In one embodiment, the cell compositions of the present invention are administered by i.v. injection. The compositions of cells may be injected directly into a transplant site, lymph node, or site of infection.

In various embodiments of the present invention, cells activated and expanded using the methods described herein, or other methods known in the art where cells are expanded to therapeutic levels, are administered to a patient in conjunction with (e.g., before, simultaneously or following) any number of relevant treatment modalities including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In some embodiments, the cells of the invention may be used in a treatment regimen in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation. Drugs that inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993) can also be used. In one embodiment, the cell compositions of the present invention are administered to a patient in conjunction with (e.g., before, simultaneously or following) bone marrow transplantation, cell ablative therapy using either chemotherapy agents such as, fludarabine, external-beam radiation therapy (XRT), cyclophosphamide, or antibodies such as OKT3 or CAMPATH. In one embodiment, the cell compositions of the present invention are administered following B-cell ablative therapy such as agents that react with CD20, e.g., Rituxan.

In one embodiment, subjects may undergo leukapheresis, wherein leukocytes are collected, enriched, or depleted ex vivo to select and/or isolate the cells of interest, e.g., T cells. These cell isolates may be expanded by methods known in the art and treated such that one or more constructs of the invention may be introduced, thereby creating a modified immune cell of the invention. Subjects in need thereof may subsequently undergo standard treatment with high dose chemotherapy followed by peripheral blood stem cell transplantation. In various embodiments, following or concurrent with the transplant, subjects receive an infusion of the expanded modified immune cells of the present invention. In one embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances, larger doses of up to 40 mg per day may be used (described in U.S. Pat. No. 6,120,766).

### EMBODIMENTS

The following causes describe particular Embodiments of the invention.

Embodiment 1 is a method of producing a Chimeric Antigen Receptor (CAR)-expressing immune cell comprising introducing a first nucleic acid sequence encoding a CAR and a second nucleic acid sequence encoding a mechanosensitive ligand to an immune cell.

Embodiment 2 is the method of embodiment 1, wherein the immune cell is selected from a T cell, NK cell, and macrophage.

Embodiment 3 is the method of embodiment 1 or embodiment 2, wherein the immune cell is a T cell.

Embodiment 4 is the method of any one of embodiments 1-3, wherein the first nucleic acid sequence encodes a CAR that binds to a tumor-specific antigen.

Embodiment 5 is the method of embodiment 4, wherein the first nucleic acid sequence encodes a CAR that binds CD19, BCMA, CD123, or Mesothelin.

Embodiment 6 is the method of any one of embodiments 1-5, wherein the first nucleic acid sequence encodes a CAR molecule comprising a CD8 leader sequence, a CD19-scFv, a CD28-hinge region, a transmembrane domain derived from CD28 and/or 4-1BB, and a cytoplasmic signaling domain of CD3ζ.

Embodiment 7 is the method of any one of embodiments 1-6, wherein the second nucleic acid sequence encodes a mechanosensitive ligand selected from the group consisting of Piezo1, a fragment thereof, and a variant thereof.

Embodiment 8 is the method of embodiment 7, wherein the second nucleic acid sequence encodes the ΔL15-20 extracellular loop and/or F2114 residue from Piezo1.

Embodiment 9 is the method of any one of embodiments 1-8, wherein the second nucleic acid sequence encodes an amino acid sequence comprising SEQ ID NO:1.

Embodiment 10 is the method of any one of embodiments 1-9, wherein the second nucleic acid sequence encodes an amino acid sequence comprising SEQ ID NO:2 and/or SEQ ID NO:3.

Embodiment 11 is the method of any one of embodiments 1-10, wherein the method comprises contacting the immune cell with a first nucleic acid molecule comprising the first sequence, and a second nucleic acid molecule comprising the second sequence.

Embodiment 12 is the method of any one of embodiments 1-10, wherein the method comprises contacting the immune cell with a nucleic acid molecule comprising the first and second nucleic acid sequence.

Embodiment 13 is the method of any of embodiments 10-12, wherein the second nucleic acid sequence comprises SEQ ID NO:4.

Embodiment 14 is the method of any of embodiments 10-12, wherein the second nucleic acid sequence comprises SEQ ID NO:5 and/or SEQ ID NO:6.

Embodiment 15 is the method of embodiment 12, wherein the nucleic acid molecule comprises a nucleic acid sequence encoding a fusion protein comprising a CAR and a mechanosensitive ligand linked by a self-cleaving peptide.

Embodiment 16 is the method of embodiment 15, wherein the self-cleaving peptide comprises P2A.

Embodiment 17 is the method of embodiment 15 or 16, wherein the nucleic acid molecule comprises a nucleic acid sequence encoding SEQ ID NO:7.

Embodiment 18 is an immune cell genetically engineered to express:
a. a Chimeric Antigen Receptor (CAR); and
b. a mechanosensitive ligand.

Embodiment 19 is the immune cell of embodiment 18, wherein the immune cell is a T cell, an NK cell, or a macrophage.

Embodiment 20 is the immune cell of embodiment 18 or embodiment 19, wherein the immune cell is a T cell.

Embodiment 21 is the immune cell of any one of embodiments 18-20, wherein the CAR binds to a tumor-specific antigen.

Embodiment 22 is the immune cell of embodiment 21, wherein the CAR binds to an antigen selected from the group consisting of CD19, BCMA, CD123, or Mesothelin.

Embodiment 23 is the immune cell of any one of embodiments 18-22, wherein the CAR comprises a CD8 leader sequence, a CD19-scFv, a CD28-hinge region, a transmembrane domain derived from CD28 and/or 4-1BB, and a cytoplasmic signaling domain of CD3ζ.

Embodiment 24 is the immune cell of any one of embodiments 18-23, wherein the mechanosensitive ligand is selected from the group consisting of Piezo1, a fragment thereof, or a variant thereof.

Embodiment 25 is the immune cell of embodiment 24, wherein the mechanosensitive ligand comprises the ΔL15-20 extracellular loop and/or F2114 residue from Piezo1.

Embodiment 26 is the immune cell of any one of embodiments 18-24, wherein the mechanosensitive ligand comprises the amino acid sequence of SEQ ID NO:1.

Embodiment 27 is the immune cell of any one of embodiments 18-25, wherein the mechanosensitive ligand comprises the amino acid sequence of SEQ ID NO:2 and/or SEQ ID NO:3.

Embodiment 28 is a population of CAR-expressing immune cells comprising a plurality of the CAR-expressing immune cells of any one of embodiments 18-27.

Embodiment 29 is a composition comprising an immune cell of any one of embodiments 18-27, or the population of immune cells of embodiment 28.

Embodiment 30 is a method of treating or preventing cancer in a subject comprising administering to the subject an effective amount of the composition of embodiment 29.

Embodiment 31 is the method of embodiment 30, wherein the cancer is selected from the group consisting of leukemia, pancreatic cancer, and breast cancer.

Embodiment 32 is a nucleic acid molecule comprising:
a. a first nucleic acid sequence encoding a CAR; and
b. a second nucleic acid sequence encoding a mechanosensitive ligand.

Embodiment 33 is the nucleic acid molecule of embodiment 32, wherein the first nucleic acid sequence encodes a CAR that binds a tumor-specific antigen.

Embodiment 34 is the nucleic acid molecule of embodiment 33, wherein the first nucleic acid sequence encodes a CAR that binds an antigen selected from the group consisting of CD19, BCMA, CD123, and Mesothelin.

Embodiment 35 is the nucleic acid molecule of any one of embodiments 32-34, wherein the first nucleic acid sequence encodes a CAR molecule comprising a CD8 leader sequence, a CD19-scFv, a CD28-hinge region, a transmembrane domain derived from CD28 and/or 4-1BB, and a cytoplasmic signaling domain of CD3ζ.

Embodiment 36 is the nucleic acid molecule of any one of embodiments 32-35, wherein the second nucleic acid sequence encodes a mechanosensitive ligand selected from the group consisting of Piezo1, a fragment thereof, or a variant thereof.

Embodiment 37 is the nucleic acid molecule of embodiment 36, wherein the second nucleic acid sequence encodes the ΔL15-20 extracellular loop and/or F2114 residue from Piezo1.

Embodiment 38 is the nucleic acid molecule of any one of embodiments 32-36, wherein the second nucleic acid sequence encodes an amino acid sequence of SEQ ID NO:1.

Embodiment 39 is the nucleic acid molecule of any one of embodiments 32-37, wherein the second nucleic acid sequence encodes an amino acid sequence of SEQ ID NO:2 and/or SEQ ID NO:3.

Embodiment 40 is the nucleic acid molecule of any of embodiments 32-38, wherein the second sequence comprises SEQ ID NO:4.

Embodiment 41 is the nucleic acid molecule of any of embodiments 32-39, wherein the second sequence comprises SEQ ID NO:5 and/or SEQ ID NO:6.

Embodiment 42 is the nucleic acid molecule of any of embodiments 32-41, further comprising a nucleic acid sequence encoding a self-cleaving peptide.

Embodiment 43 is the nucleic acid molecule of embodiment 42, wherein the self-cleaving peptide comprises P2A.

Embodiment 44 is the nucleic acid molecule of embodiment 43, wherein the self-cleaving peptide comprises an amino acid sequence of SEQ ID NO:7.

Embodiment 45 is the nucleic acid molecule of any of embodiments 32-44, wherein the nucleic acid molecule encodes a fusion protein comprising a CAR and a mechanosensitive ligand linked by a self-cleaving peptide.

Embodiment 46 is the nucleic acid molecule of any of embodiments 32-45, wherein the nucleic acid molecule comprises a nucleic acid sequence of SEQ ID NO:8.

Embodiment 47 is the nucleic acid molecule of any of embodiments 32-46, wherein the nucleic acid molecule comprises a nucleic acid sequence of SEQ ID NO:8 linked to a nucleic acid sequence encoding a CAR.

Embodiment 48 is a composition comprising a first nucleic acid molecule and a second nucleic acid molecule, wherein the first nucleic acid molecule comprises a nucleic acid sequence encoding a CAR, and the second nucleic acid molecule comprises a nucleic acid sequence encoding a mechanosensitive ligand.

Embodiment 49 is the composition of embodiment 48, wherein the first nucleic acid molecule encodes a CAR that binds a tumor-specific antigen.

Embodiment 50 is the composition of embodiment 49, wherein the first nucleic acid molecule encodes a CAR that binds an antigen selected from the group consisting of CD19, BCMA, CD123, and Mesothelin.

Embodiment 51 is the composition of any one of embodiments 48-50, wherein the first nucleic acid molecule encodes a CAR molecule comprising a CD8 leader sequence, a CD19-scFv, a CD28-hinge region, a transmembrane domain derived from CD28 and/or 4-1BB, and a cytoplasmic signaling domain of CD3ζ.

Embodiment 52 is the composition of any one of embodiments 48-51, wherein the second nucleic acid molecule encodes a mechanosensitive ligand selected from the group consisting of Piezo1, a fragment thereof, and a variant thereof.

Embodiment 53 is the composition of embodiment 52, wherein the second nucleic acid molecule encodes the ΔL15-20 extracellular loop and/or F2114 residue from Piezo1.

Embodiment 54 is the composition of any one of embodiments 48-52, wherein the second nucleic acid molecule encodes an amino acid sequence comprising SEQ ID NO:1.

Embodiment 55 is the composition of any one of embodiments 48-54, wherein the second nucleic acid molecule encodes an amino acid sequence comprising SEQ ID NO:2 and/or SEQ ID NO:3.

Embodiment 56 is the composition of any of embodiments 48-54, wherein the second nucleic acid molecule comprises a nucleic acid sequence of SEQ ID NO:4.

Embodiment 57 is the composition of any of embodiments 48-55, wherein the second nucleic acid molecule comprises a nucleic acid sequence of SEQ ID NO:5 and/or SEQ ID NO:6.

Embodiment 58 is a composition comprising at least one nucleic acid molecule, wherein the composition comprises:
a) a first nucleic acid sequence encoding a CAR; and
b) a second nucleic acid sequence encoding a mechanosensitive ligand.

Embodiment 59 is the composition of embodiment 58, wherein the composition comprises a nucleic acid molecule comprising the first nucleic acid sequence and the second nucleic acid sequence.

Embodiment 60 is the composition of embodiment 58 or embodiment 59, wherein the composition comprises a first nucleic acid molecule and a second nucleic acid molecule, wherein the first nucleic acid molecule comprises a nucleic acid sequence encoding a CAR, and the second nucleic acid molecule comprises a nucleic acid sequence encoding a mechanosensitive ligand.

Embodiment 61 is the composition of any one of embodiments 58-60, wherein the first nucleic acid sequence encodes a CAR that binds an antigen selected from the group consisting of CD19, BCMA, CD123, and Mesothelin.

Embodiment 62 is the composition of any one of embodiments embodiment 58-61, wherein the first nucleic acid sequence encodes a CAR molecule comprising a CD8 leader sequence, a CD19-scFv, a CD28-hinge region, a transmembrane domain derived from CD28 and/or 4-1BB, and a cytoplasmic signaling domain of CD3ζ.

Embodiment 63 is the composition of any one of embodiments 58-62, wherein the second nucleic acid sequence encodes a mechanosensitive ligand selected from the group consisting of Piezo1, the ΔL15-20 extracellular loop of Piezo1, the F2114 residue from Piezo1, a fragment thereof, and a variant thereof.

Embodiment 64 is the composition of any one of embodiments 58-63, wherein the second nucleic acid sequence encodes an amino acid sequence of SEQ ID NO:1, SEQ ID NO: 2, SEQ ID NO:3, or a combination thereof.

Embodiment 65 is the composition of any one of embodiments 58-64, wherein the second nucleic acid sequence comprises SEQ ID NO:4, SEQ ID NO:5, SEQ ID NO:6, or a combination thereof

Embodiment 66 is the composition of embodiment 59, wherein the nucleic acid molecule encodes a fusion protein comprising a CAR and a mechanosensitive ligand linked by a self-cleaving peptide.

Embodiment 67 is the composition of embodiment 59, wherein the nucleic acid molecule comprises a nucleic acid sequence of SEQ ID NO:8 linked to a nucleic acid sequence encoding a CAR.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore specifically point out exemplary embodiments of the present invention, and are not to be construed as limiting in any way the remainder of the disclosure.

### Example 1: Investigating Piezo1-Mediated Mechanotransduction Machinery in CAR Signaling

With the recent FDA approval of chimeric antigen receptor (CAR) T-cell therapies for B-cell malignancies, CAR T-cell therapy has been remarked as a promising strategy to cure relapsed and refractory leukemia. However, the clinical benefit of CAR T-cell therapy varies tremendously in different clinical trials, and overall patient responses remain unfavorable. Current research has been mostly focused on identifying new antigens and designing new CAR immunoreceptors and co-receptors that can be effectively activated. However, apart from these biological factors, critical biophysical regulatory factors and mechanisms may also be involved in regulating CAR T-cell function, which remain largely unexplored. After antigen recognition through CAR immunoreceptors, CAR T-cell activation is also cooperated by an intense retrograde flow of cytoskeletons that transduce transient force signals to develop a mature immune synapse between the CAR T and target cancer cells to facilitate a robust cytolytic killing process. The variable clinical responses may arise from the initial quality of patient T-cells, CAR T-cell manufacturing process as well as in vivo T-cell exhaustion and antigen escape, all are possibly associated with changes in CAR T-cell biomechanics such as dispersed cytoskeleton networks, weakened force response and mechanotransduction signaling strength. By targeting these mechano-regulatory mechanisms, the delicate control of CAR-mediated mechanical forces during CAR T-cell activation and cytotoxic processes will provide valuable opportunities to improve the efficacy of CAR T-cell therapy.

Therefore, a new generation of “mechano-enhanced” CAR T-cells are engineered herein to effectively enhance CAR T-cell contractility, activation and cytotoxicity.

Specifically, for a more effective and sustained immunotherapy outcomes, a new generation of CAR T-cells with an integrated mechanosensitive structure are developed to enhance the mechanosensitivity of CAR T-cells, rendering them more responsive to force-driven immunological synapse formation, activation, and cytotoxic processes. Modulation of Piezo1-mediated signaling pathways within CAR T-cells are targeted. The therapeutic efficacy of these new CAR T-cell products are validated both with in vitro Leukemia-on-a-Chip model and in vivo mouse model.

To date, studies on the fundamental regulatory mechanisms of CAR T-cell mechanotransduction and their links to CAR T-cell effectiveness and persistence have been scarce. These regulatory mechanisms are investigated herein, revealing that Piezo1 ion channel, in conjunction with actomyosin effectors, collaboratively governs mechanotransduction, orchestrating CAR T-cell activation, immunological synapse (IS) formation, and immune responses.

### Mapping the Synaptic Force Dynamics of CAR T-Cells Upon CD19 Activation.

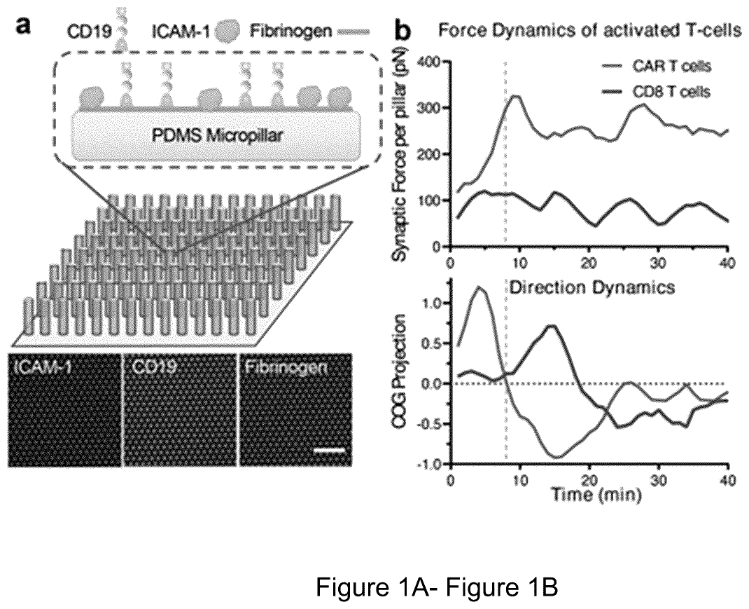

The subcellular synaptic force dynamics (both the magnitude and direction) of single 4-1BBζ-CAR T-cells upon CD19 activation during IS formation were measured using an elastic polydimethylsiloxane (PDMS) micropillar array force sensor (Shao and Fu, Adv Mater, 2014. 26(10): p. 1494-533) functionalized with CD19 antigen and intercellular adhesion molecule 1 (ICAM-1). The elastic micropillars (height of 6 μm, diameter of 0.8 μm) are fabricated from PDMS using soft lithography technique with a double molding process. The temporal analysis of force responses revealed that the synaptic force exhibited a biphasic feature (FIG. 1b). After encountering CD19 and ICAM-1, CAR T-cells exerted outward pushing forces and the forces increased in the adhesion and spreading period (0-8 min), then soon transit into inward contractile forces in CAR centralization period (8-40 min). Further, the synaptic forces generated by CD19-activated CAR T-cells are greater in magnitude and showed faster rate than that of conventional TCR-activated in CD8+ T-cells (FIG. 1b). These findings unveiled a distinctive biophysical mechanism guiding CD19-mediated activation in CAR T-cells, comparing to the conventional TCR-mediated activation in T-cells.

### Dissecting the Mechanotransductive Signaling Mechanisms in CAR T-Cell Activation

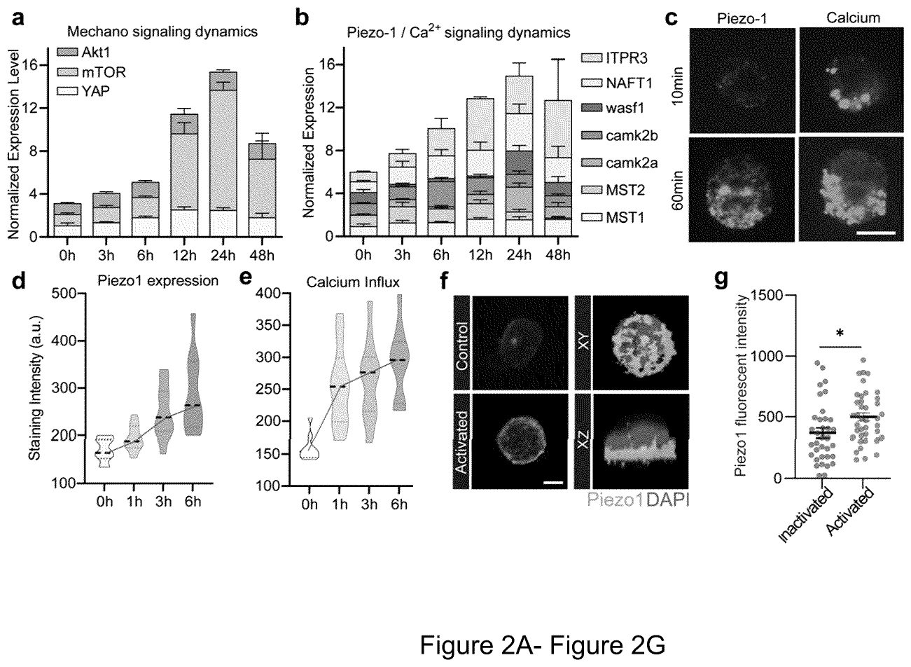

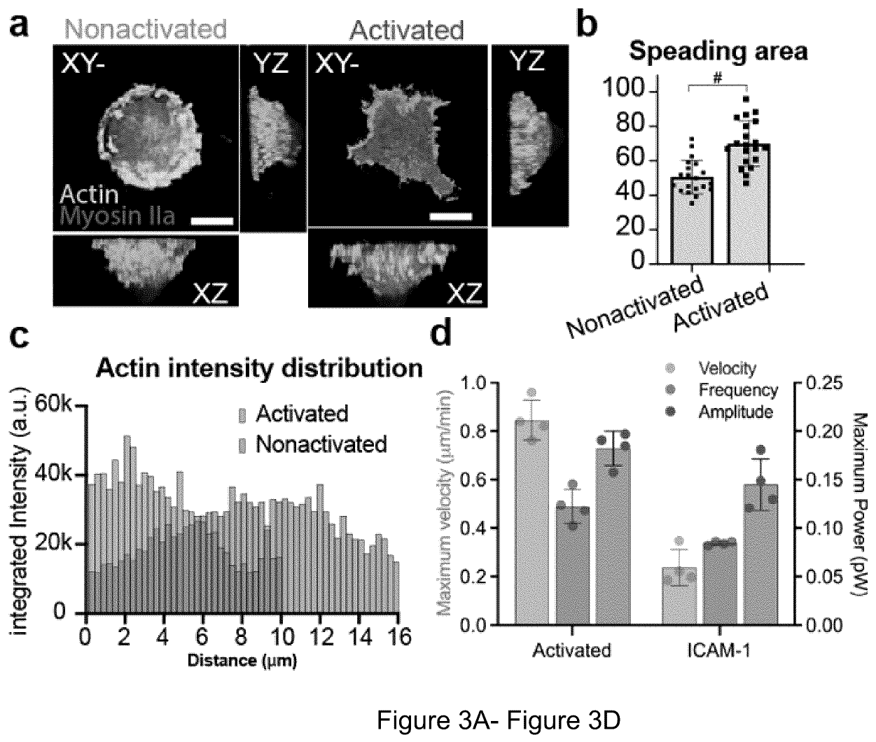

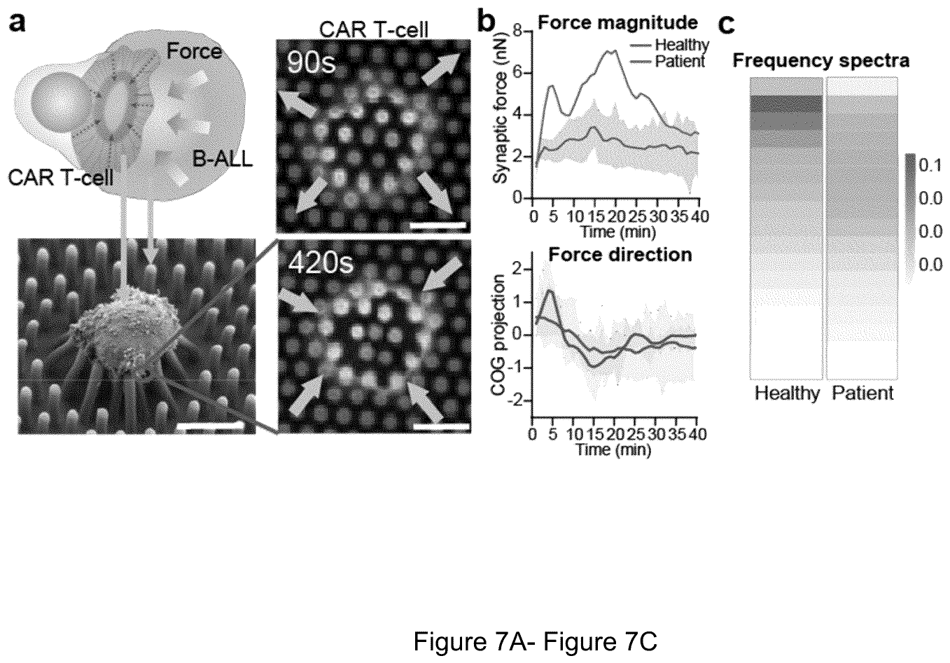

There have been limited studies investigating the major regulatory molecular mechanisms of CAR T-cell mechanotransduction. RT-qPCR analysis demonstrated upregulations of genes associated with major mechanotransductive signaling in activated CAR T-cells, such as YAP/TAZ, PI3K/Akt, canonical and noncanonical Wnt (FIG. 2a), and Piezo1/Ca²⁺ (FIG. 2b). Among these regulatory signaling, intracellular Ca²⁺ signaling (ITPR3, NFAT1) are most obviously upregulated candidates, indicating the essential roles of Piezo1-mediated Ca²⁺ signaling during CAR T-cell activation. Consistent with the gene analysis, immunostaining results showed that Piezo1 expression was significantly enhanced in activated CAR T-cells (FIG. 2c and FIG. 2d). Intracellular Ca²⁺ levels measured by Fluo4-AM Calcium indicator were also elevated upon CD19 activation (FIG. 2c and FIG. 2e). The z-scan image of the activated T-cells also suggested that Piezo1 is more concentrated at the interface of T-cells and the antibody-coated substrate during activation (FIG. 3f). Patient-derived CAR T-cells have relative lower expression of Piezo1 compared to healthy ones, explaining the less robust mechanical responses upon CD19 activation (FIG. 7).

### Cytoskeletal Machinery Regulating CAR T-Cell Force Dynamics and IS Formation.

The formation and maintenance of IS in CAR T-cells rely on (1) actin cytoskeleton remodeling for spreading and adhesion of the T-cell over the cancer cell surface, (2) elevated cytoskeletal forces for stabilizing the IS and clustering of signaling molecules, and (3) mechanotransduction generated by the cytoskeleton to trigger downstream signaling for CAR T-cell activation. Unlike the classical TCR, the nonclassical CAR IS is less organized structurally, with diffuse CAR clustering, and a reduced distribution of actin in the distal CAR IS (Davenport et al., PNAS, 2018. 115(9): p. E2068-E2076). Thus, how the cytoskeletal machinery supports the unique nature of mechanotransduction in CAR IS is explored herein. First, actin cytoskeleton remodeling during IS formation in CAR T-cells is monitored using a SPY650-FastAct™ live actin probe. The dynamics of F-actin organization, myosin activity, and LFA-1 are monitored, which are all essential cytoskeleton remodeling during IS formation (FIG. 3). Moreover, the activity of potentially IS related markers upon CAR activation are monitored by immunostaining, such as phosphorylation of proximal Lek and ZAP-70 and distal recruitment of ERK proteins. During the process of IS formation, F-actin within the IS undergoes robust retrograde flow, which is driven by both actin polymerization and myosin-based contractility (Basu and Huse, Trends in cell biology, 2017. 27(4): p. 241-254).

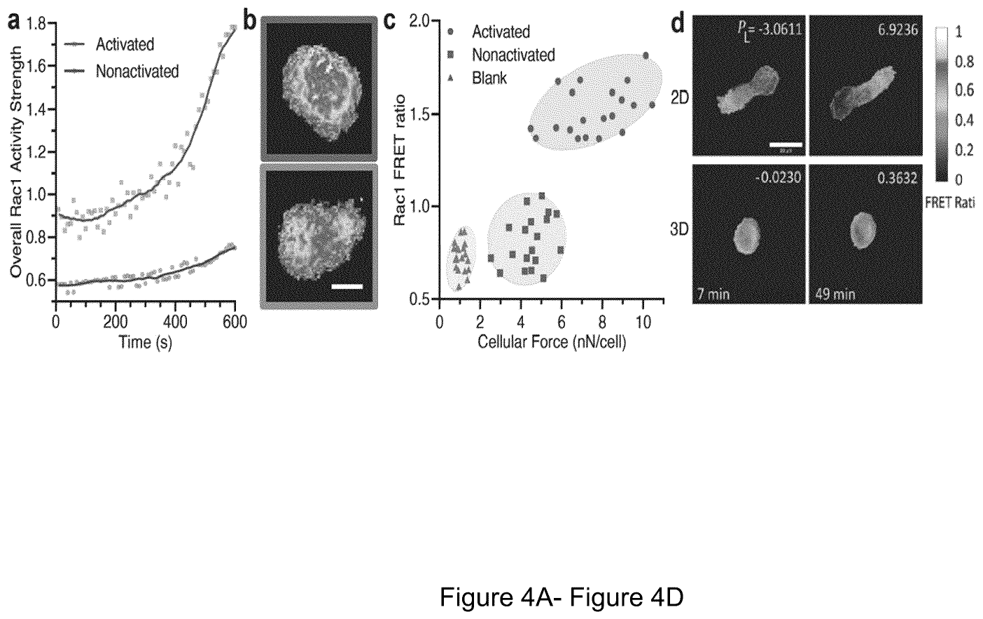

How Rac1 and RhoA GTPases regulate force transmission and cytoskeletal remodeling during CAR T-cell activation and IS formation is elucidated using genetically encoded Rac1 and RhoA FRET mechano-biosensors (Miskolci et al., J Immuno, 2018, 196(8):3879-93; Spiering et al., Methods Cell Biol, 2013, 114:593-609) (FIG. 4). In response to calcium signaling, Rac1 initiates rapid actin filament turnover which facilitates the spreading and adhesion of CAR T-cells on target cancer cells. RhoA acts as a key signaling molecule that triggers actomyosin contraction through the activation of ROCK and subsequent phosphorylation of the myosin light chain (Methods Cell Biol, 2013, 114:593-609). In detail, naïve CAR T-cells are transfected with a plasmid encoding Rac1 or RhoA FRET biosensors one day before they are seeded onto the functionalized micropillar substrates to simultaneously monitor the time-lapse synaptic force and the accompanying subcellular Rac1 and RhoA activities with live cell imaging. Rac1 coordinates cytoskeleton remodeling in the distal CAR IS in the spreading phase (phase I), while RhoA activities are significantly enhanced during CAR centralization (phase II) to facilitate contraction force generation in activated CAR T-cells. Similar to the force dynamics analysis, spatiotemporal activities of actin, RhoA and Rac1 GTPases are analyzed with HHT spectral decomposition of the live cell data into frequency spectra (FIG. 3, Ma et al., PLoS computational biology, 2018, 14.8). Various CAR T-cell mechanical phenotypes share similar mechanotransduction circuitry pattern, but their mechanotransduction activity levels differ significantly. The frequency shift is quantified to serve as a sensitive marker of the CAR T-cell mechanosensitivity.

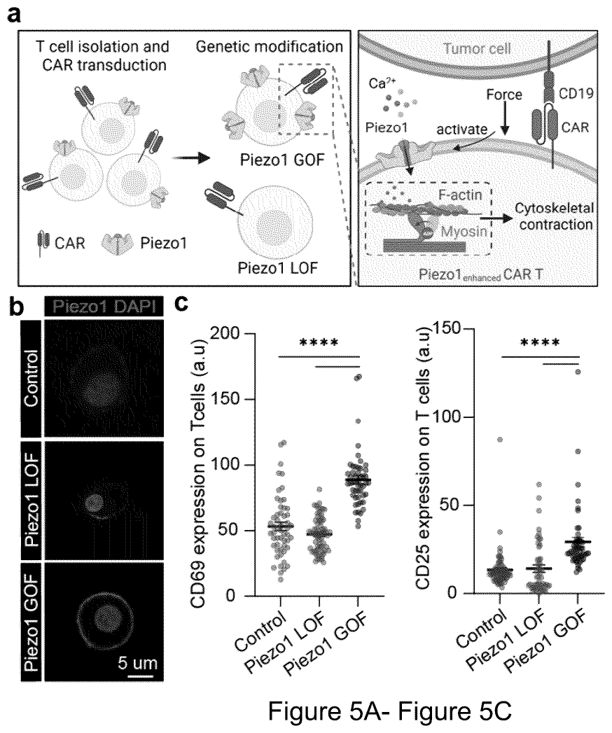

Preliminary study using immunostaining showed concentrated actin and myosin at the IS interface in activated CAR T-cell (FIG. 5). The intricate cytoskeletal machinery governing the dynamic force response during CAR T-cell activation and IS formation was evaluated. A Rac1 FRET biosensor was used to probe the activity of the GTPase Rac1 protein, a key regulator of actin cytoskeletal rearrangement, during CD19-mediated activation of CAR T-cells (FIG. 4). These results demonstrated that Rac1 activities increase in activated CAR T-cells compared to that of nonactivated ones (FIG. 4a and FIG. 4b). Meanwhile, the robust Rac1 activity is positively correlated to the increase of synaptic force (FIG. 4c).

To investigate the role of each signaling pathway in CAR T-cell activation and mechanotransduction, gain-of-function (GOF) and loss-of-function (LOF) approaches are used. The relative importance of each pathway is assayed by including Piezo1 inhibitor (GsMTx4) and stimulator (Yodal), Rac1 inhibitor (NSC23766) and activator (CN04), ROCK inhibitor (Y27632) and RhoA stimulator (LPA) in functional assays.

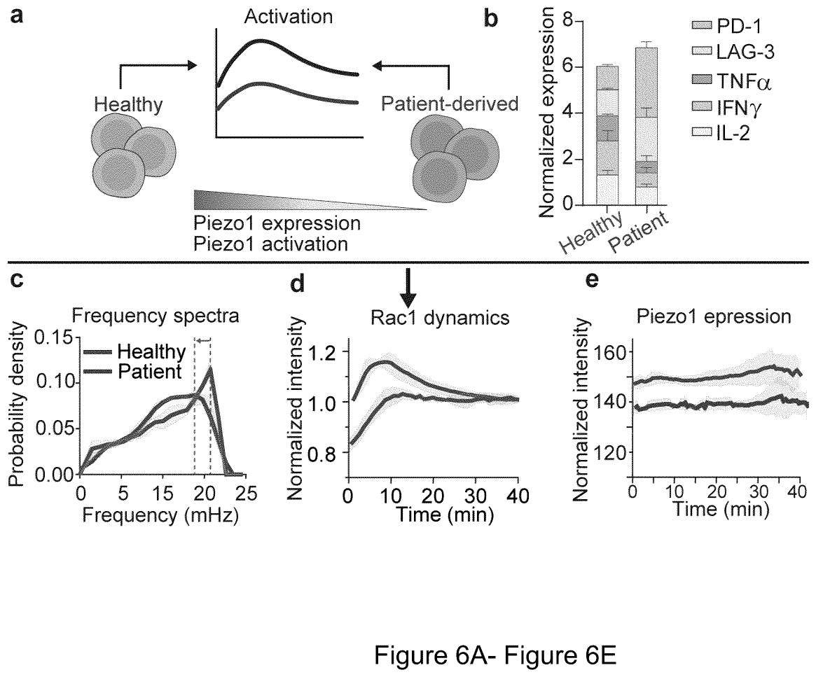

In addition to pharmaceutic modulation, Piezo1 expression is genetically modulated in CAR T-cells using Piezo1-specific CRISPR plasmids for gene knockout and activation of Piezo1, and examine how this impacts Ca2+ influx, Rac1 activity, IS formation, and synaptic force generation (FIG. 6a). In the Piezo1 gain-of-function (Piezo1 GOF) CAR T-cells, a CRISPR activation plasmid integrating a synergistic activation mediator activation system is used to specifically upregulate gene expression of Piezo1. The Piezo1 loss-of-function (Piezo1 LOF) CAR T-cells are transduced with a CRISPR/Cas9 Knockout Plasmid consisting of the Cas9 nuclease and the PIEZO1-specific gRNA was designed for maximum knockout efficiency.

How Piezo1 affects the phenotypical and mechanical signatures of CAR T-cell is tested by analyzing the above-mentioned CAR T-cell function, force and signaling. Reduced Piezo1-mediated Ca2+ signaling weakens dysfunctional CAR T-cells' ability to generate sufficient mechanical cues for an immune response. Enhanced Piezo1 activation in Piezo1 GOF cells (FIG. 6b), along with its downstream Rac1 and RhoA/ROCK/LIM-kinase axis, greatly promotes the CAR activation (FIG. 6c) and synaptic force. Enhanced Piezo1 in CAR T-cells elevates proximal Lek and ZAP-70 phosphorylation, distal ERK recruitment, accompanied with promoted Rac1 and RhoA/ROCK signaling activities and synaptic force exertion during IS formation. In contrast, Piezo1 LOF cells shows reduced activation (FIG. 6c) and Rac1 activities, thus compromises CAR T-cells' ability to support synaptic force transmission during for IS formation. Pharmaceutic inhibition of Rac1 or RhoA/ROCK GTPases attenuates CAR T-cells' cytoskeleton remodeling and mechanotransduction, while these GTPases activators (CN04, LPA) increase the mechanotransduction strength and force generation. Through these detailed investigations, the unique complex networks between Piezo1-driven Ca2+ signaling, Rac1/RhoA activity, and synaptic force dynamics in CAR T-cell mechanotransduction are unraveled.

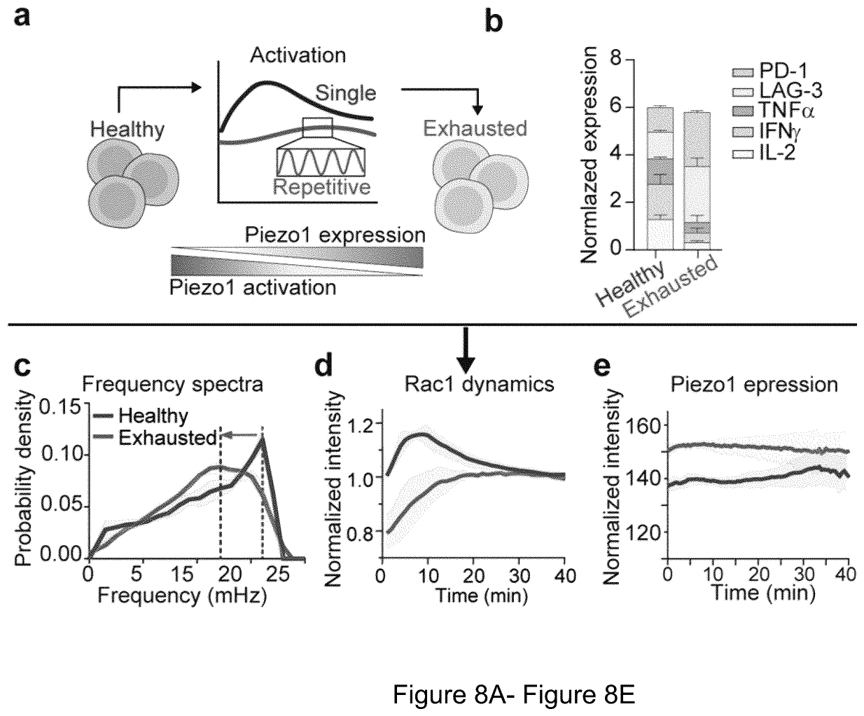

The assay is expanded to patient-derived CAR T-cell product to further validate the role of Piezo1 signaling pathways in regulating the effectiveness of clinical immune responses, regarding synaptic force dynamics, Rac1 activities, and Piezo1/Ca2+ activity levels upon CD19 activation. Patient-derived CAR T-cells with low Piezo1 expression and activation, show significantly low yet transient Ca2+ influx, less dynamic Rac1 and RhoA activities (FIG. 7), and a diminished synaptic force response (FIG. 8), which attribute to their suppressive immune responses.

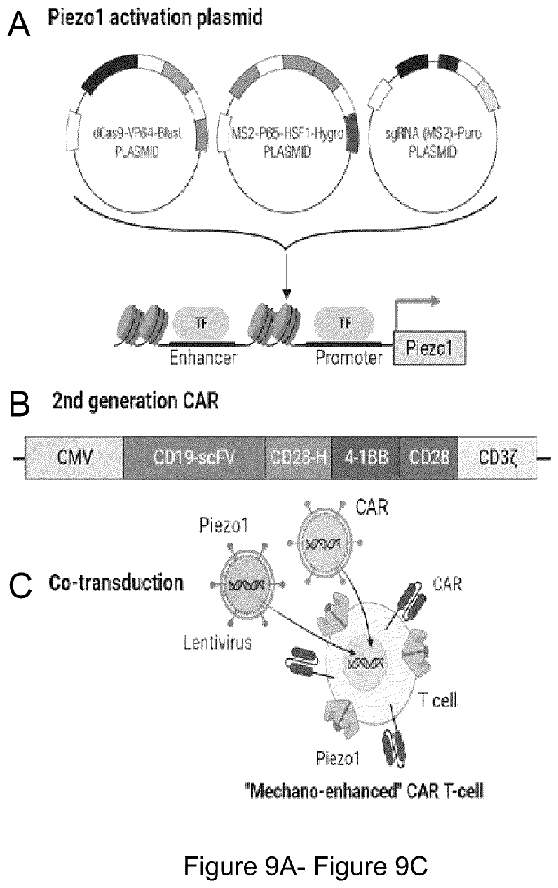

Moreover, previous study demonstrated that excessive calcium level triggers exhaustion via NFAT signaling and glycolysis. Continuous antigen stimulation ultimately leads to hyperactive and unsynchronized Piezo1 activation in exhausted CAR T-cells, impairing both the mechano- and immune response in CAR T-cells. Exhausted CAR T-cells show a delayed and prolonged Ca2+ influx, thus resulting in less robust cytoskeletal dynamics and a diminished force response (FIG. 9).

To study how CAR T-cell exhaustion influence cell force and mechanotransduction CAR T-cells were continuously stimulated with activating dynabeads containing CD3p/CD28 antibodies for 8 days to induce T-cell exhaustion (FIG. 8a, Jenkins et al., Oxford Open Immunology, 2023. 4(1): p. iqad006; Vardhana et al., Nature immunology, 2020. 21(9): p. 1022-1033; Belk et al., Cancer Cell, 2022. 40(7): p. 768-786. e7). Increases in the exhaustion makers of PD-1 and LAG-3 and decreases in expression levels of immune functional cytokines IFNγ and TNFα were observed in CAR T-cells upon CD19 activation (FIG. 8b). Piezo1 activities in the exhausted CAR T-cells were abnormally upregulated due to continuous activation. As a result, exhausted CAR T-cells had a hindered level of Rac1 activity and delayed force dynamics upon CD19 activation when compared to healthy CAR T-cells (FIG. 8c-FIG. 8e).

### Example 2: Engineering a New Generation of “Mechano-Enhanced” CAR T-Cells for Efficient Immunotherapy

Precisely modulating CAR T-cells' mechanosensitivity holds the potential to sustain T-cell activation and enhance cytotoxic activities, thereby increasing their killing efficiency and persistence. To date, current CAR designs predominantly focus on promoting or inhibiting the immunological and biochemical attributes of CAR T-cells. This study pivots towards manipulating the intrinsic mechanical signaling of CAR T-cells via a “mechanogenetic” approach. As a “mechanical promoter,” regulating Piezo1 activity could significantly increase mechanosensation and mechanotransduction of CAR T-cells. Thus a new generation of “mechano-enhanced” CAR are designed with a mechanosensitive Piezo1 domain as a mechanical promoter that facilitates CAR T-cell activation and cytotoxicity.

Design A. Engineering a Novel CAR T-Cell with Enhanced Mechanosensitivity Via Co-Transduction with CAR and Piezo1 Lentiviral Vectors.

Piezo1 construct: PIEZO1 overexpression lentiviral construct are designed to specifically upregulate Piezo1 gene expression (Santa Cruz). Both CAR and Piezo1 lentiviral constructs contain GFP and mCHERRY enabling enrichment by flow cytometry (FIG. 9a).

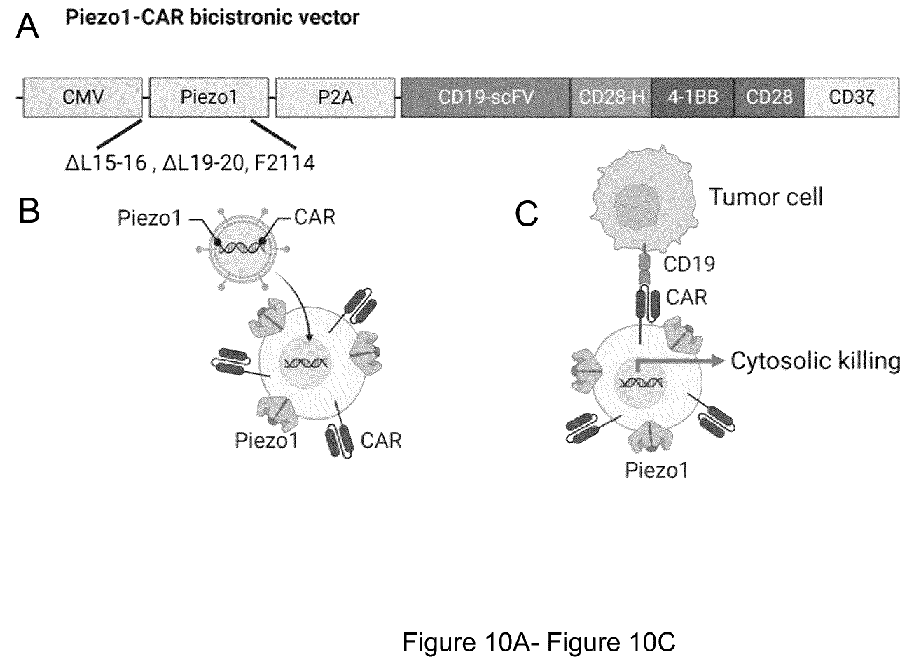

CAR construct: a standard CD19 4-1BBζ-CAR construct containing an antigen recognition domain (scFv), CD28 hinge, transmembrane domain CD28, 4-1BB co-stimulatory domain, and CD3ζ activation domain (FIG. 10b).

Manufacturing of Piezo1-CAR T-cell (FIG. 9c): CD4+ and CD8+ T-cells isolated from healthy donors' or patients' PBMCs are first activated using Dynabeads™ Human T-Activator CD3/CD28 (Gibco) overnight, then co-transduced with CAR and Piezo1 lentiviral particles. These cells are expanded in culture media supplemented with IL-2. On day 7, CAR (GFP+) and Piezo1 (mCHERRY) double positive cells will be isolated by flow cytometry.

Design B. Engineering a “Mechano-Enhanced” Piezo1-CAR T-Cell with Via Bicistronic CAR-Piezo1 Vector.

Piezo1 exhibits a unique three-bladed, propeller-like electron cryo-microscopy structure, comprising a substantial 2,547 amino acids in humans (Wang et al., Biophysical Journal, 2018. 114(3): p. 113a-114a). The considerable size of Piezo1 poses a challenge for efficient lentiviral packaging (max 10 kb capacity) due to limitations in cargo size. To address this challenge, instead of incorporating the full-length Piezo1 protein in the same lentiviral plasmid with CAR, an abbreviated Piezo1 protein is designed which is significantly smaller in size but meticulously preserve the critical mechanosensitive function of Piezo1 in CAR T-cells.

Previous studies have indicated that while the central pore forms the ion-conducting pathway, the curved peripheral transmembrane blade (specifically, the ΔL15-20 extracellular loops) and the anchor domain (notably, the F2114 residue) are critical regions for Piezo1-mediated force transduction (Wang et al., Biophysical Journal, 2018. 114(3): p. 113a-114a; Zhao et al., Nature, 2018. 554(7693): p. 487-492; Vero et al., Channels, 2021. 15(1): p. 438-446). To engineer the “mechano-enhanced” CAR T-cells, T-cells are transfected with a bicistronic lentiviral vector containing genes encoding for both CAR and Piezo1-mimicking structure. The bicistronic vector construct contains two open reading frames separated by a 2A “self-cleaving” peptide sequence, P2A (FIG. 10a). 2A peptides were chosen as a linker for a more comparable expression of the upstream and downstream genes. An activation plasmid containing ΔL15-20, and the F2114 residue is used to insert the Piezo1 gene to the upstream of the P2A peptide, and different combination is initially tested to finalize a plasmid with optimal on-target score. Next, a standard CD19 4-1BBζ-CAR construct is cloned into the downstream of the P2A. Promoters and enhancers are added upstream of the Piezo-mimicking gene and the CAR construct to drive their expression in T-cells. The Piezo1-CAR bicistronic vector is then transfected in the T-cell population via lentiviral transduction and the positive clones selected by antibiotics are expanded for further experimentation (FIG. 10c).

Validation of Piezo1-CAR T-Cells with In Vitro Leukemia-On-a-Chip and In Vivo Mouse Models

To experimentally understand the response of Piezo1-CAR T-cells, their performance is validated using both in vitro and in vivo models, where standard CD19 4-1BBζ-CAR T-cells and unmodified T-cells as controls. Improved mechanical functions via enhanced Piezo1 signaling will increase CAR T-cell killing efficiency as well as exhaustion resistance over extended periods.

1) Functional characterization of Piezo1-CAR T-cell. To understand how the Piezo1-CAR transduction affects the immunophenotype and biological function of CAR T-cells, cell count, viability, and CD4/CD8 ratios are examined in the T-cells. To assess activation state and functional disposition, we will measure CD25, CD69, and PD1, Tim-3, as well as LAG3 expression by flow cytometry. The global transcriptomic landscape of single Piezo1-CAR T-cells (e.g., activation markers and differentiation markers) and the mechanotransduction-related pathways (i.e., actin cytoskeleton, RhoA, Rac1 signaling) are profiled using the 10× Genomics scRNA-Seq platform. To prove strengthened force-dependent CAR T-cell response, the force dynamics, Rac1-mediated cytoskeletal remodeling, Piezo1 activities and calcium signaling of the newly developed Piezo1-CAR T-cells are compared with the conventional CD19 4-1BBζ-CAR T-cells.

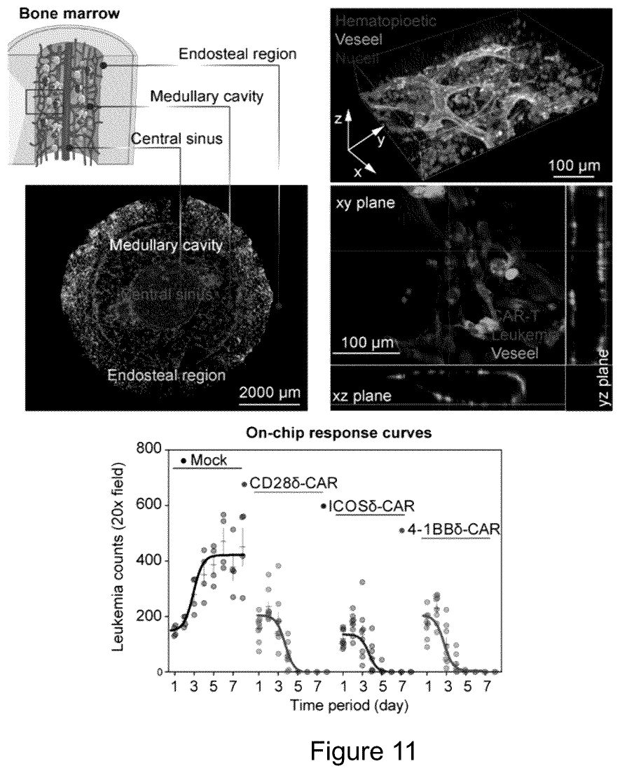

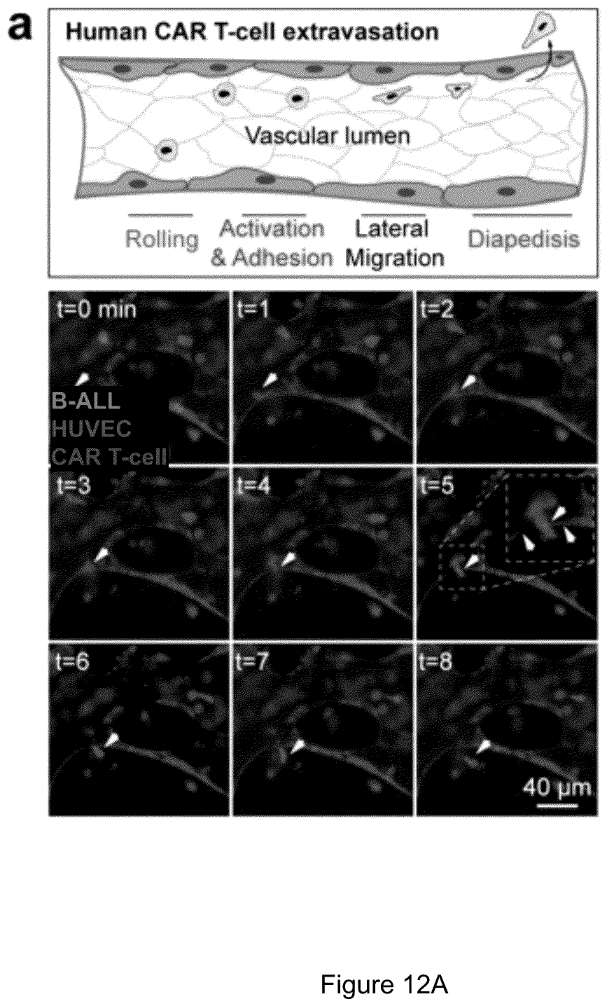

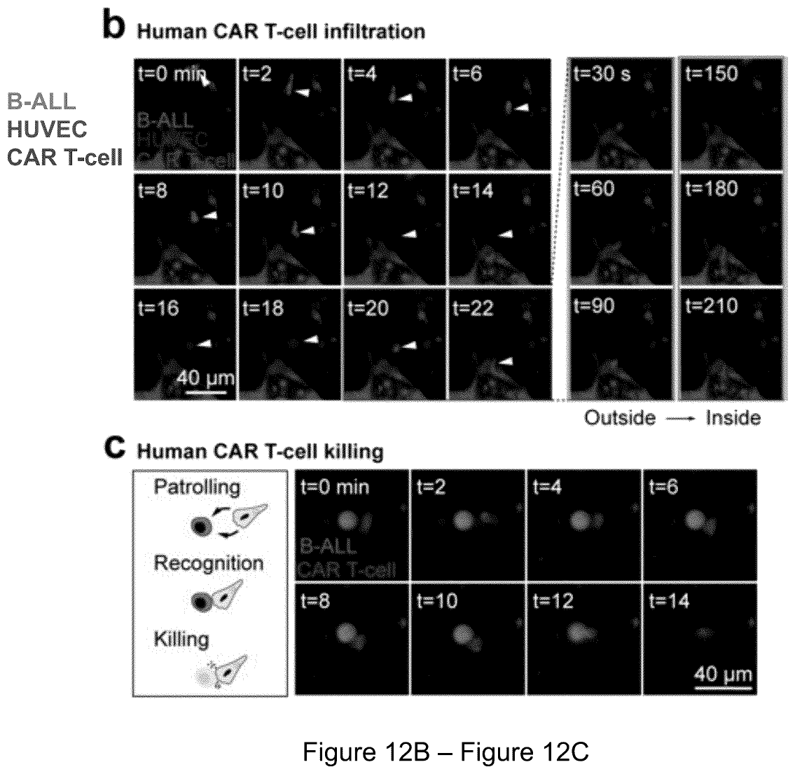

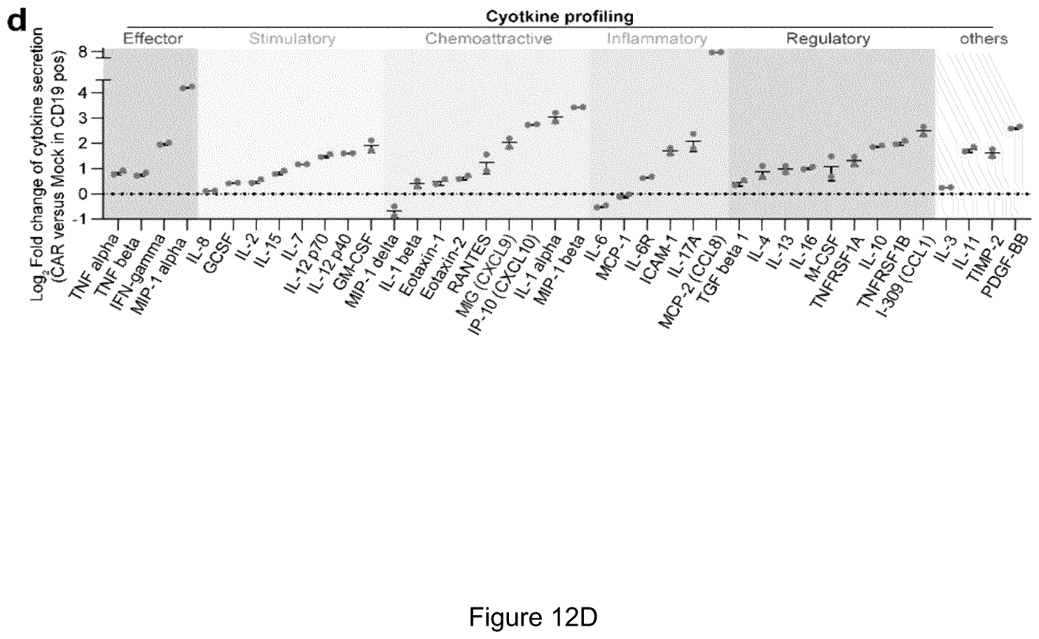

2) In vitro on-chip validation. An ex vivo immunocompetent Leukemia-on-a-Chip model has been developed, mimicking both leukemic bone marrow stromal and immune niches on-chip by populating the chip with human bone marrow mononuclear cells (STEMCELL), B-cell acute lymphoblastic leukemia (B-ALL) blasts (ATCC), stromal cells (Lonza) (FIG. 11, Ma et al., Science advances, 2020. 6(44): p. eaba5536). Following the establishment of such leukemia model, different doses (effector:tumor=1:1, 1:4 or 1:8) of Piezo1-CAR T-cells are infused into the perfusable vessels from central sinus of the leukemia chip and CAR T-cell dynamics are monitored in real-time (FIG. 12). CAR T-cell functionality, such as T-cell activation, proliferation, exhaustion, cytokine secretion, and cytotoxicity, is characterized after its interaction with leukemia blasts on-chip for 5-14 days, as described in (Ma et al., Science advances, 2020. 6(44): p. eaba5536). Specifically, a stress test is adapted with lower doses (effector:tumor=1:4 or 1:8) of CAR T-cells, showing that the Piezo1-CAR T-cells demonstrate higher cancer cell killing efficiency and persistence compared to conventional CAR design.

3) In vivo mouse model validation. To investigate the impact of co-expression of Piezo1 in CAR T-cell, an in vivo “stress test” experiment (Ghassemi et al., Nat Biomed Eng, 2022. 6(2): p. 118-128) is done using a titration of CAR T-cells to determine if enhancing Piezo1 reduces the dose requirements for efficacy compared to the standard CAR T-cell. Pre-clinical experiments are carried out in vivo using NSG mice xenografted with 1×106 NALM6 B-ALL cells co-expressing GFP and luciferase (GFP/Luc). After tumor gets established (4-5 days), animals receive a limiting dose (1-10×10⁵) of CART19-Piezo1, CART19-GFP, or NTD by intravenous injection. Tumor growth or reduction is evaluated by bioluminescent imaging by IVIS. The following functional readouts of efficacy are used to evaluate the engineered T-cells: 1) reduction of longitudinal tumor burden; 2) weight loss in animals as a measure of morbidity and 3) prolongation of overall survival. Engraftment kinetics are monitored by weekly bleeding to assay CAR T-cell abundance and phenotype. At regular intervals, peripheral blood is collected, and CAR T-cell abundance is measured by flow cytometry. Piezo1-CAR T-cells are more likely to persist in in vivo animal studies as observed in the in vitro Leukemia-on-a-Chip model. Collectively, these studies validate the potency of Piezo1-CART-cells compared to conventional CAR design.

An alternative method is co-transfection of the Piezo1 activation plasmid and CAR construct independently (FIG. 9). Further, other Piezo1-associated domains are screened to improve the Piezo1-CAR design. Engineering mechanosensitive structures in CAR T-cells targeting other signaling pathways such as non-canonical Wnt signaling, is also explored. Given the importance of serial killing for CAR T-cells, an approach to uncouple migration from cytolysis is developed which involves a Matrigel barrier separating tumor cells secreting chemoattractants and CAR T-cells. As the density of the Matrigel can be easily tuned to match solid tumor interstitial rigidities, a future direction is to determine if Piezo 1-mediated mechanotransduction can support enhanced invasion through Matrigel. These findings are immediate translational relevance for advancing CAR T-cell function in solid tumors.

The disclosures of each and every patent, patent application, and publication cited herein are hereby incorporated herein by reference in their entirety. While this invention has been disclosed with reference to specific aspects, it is apparent that some embodiments and variations of this invention may be devised by others skilled in the art without departing from the true spirit and scope of the invention. The appended claims are intended to be construed to include all such aspects and equivalent variations.

### Sequences:

| | | |
|---|---|---|
| SEQ ID NO: 1 | Amino acid | Piezo1 |
| SEQ ID NO: 2 | Amino acid | ΔL15-20 from Piezo1 |
| SEQ ID NO: 3 | Amino acid | F2114 from Piezo1 |
| SEQ ID NO: 4 | Nucleic acid | Piezo 1 |
| SEQ ID NO: 5 | Nucleic acid | ΔL15-20 from Piezo1 |
| SEQ ID NO: 6 | Nucleic acid | F2114 from Piezo1 |
| SEQ ID NO: 7 | Amino acid | P2A |
| SEQ ID NO: 8 | Nucleic acid | Bicistronic vector |

(Piezo1 AA)
SEQ ID NO: 1
RQFVKEKLLKWAESPAALTEVTVADTEPTRTQTLLQSLGELVKGVYAKYWIYVCAG
MFIVVSFAGRLVVYKIVYMFLFLLCLTLFQVYYSLWRKLLKAFWWLVVAYTMLVLI
AVYTFQFQDFPAYWRNLTGFTDEQLGDLGLEQFSVSELFSSILVPGFFLLACILQLHYF
HRPFMQLTDMEHVSLPGTRLPRWAHRQDAVSGTPLLREEQQEHQQQQQEEEEEEED
SRDEGLGVATPHQATQVPEGAAKWGLVAERLLELAAGFSDVLSRVQVFLRRLLELH
VFKLVALYTVWVALKEVSVMNLLLVVLWAFALPYPRFRPMASCLSTVWTCVIIVCK
MLYQLKVVNPQEYSSNCTEPFPNSTNLLPTEISQSLLYRGPVDPANWFGVRKGFPNLG
YIQNHLQVLLLLVFEAIVYRRQEHYRRQHQLAPLPAQAVFASGTRQQLDQDLLGCLK
YFINFFFYKFGLEICFLMAVNVIGQRMNFLVTLHGCWLVAILTRRHRQAIARLWPNY
CLFLALFLLYQYLLCLGMPPALCIDYPWRWSRAVPMNSALIKWLYLPDFFRAPNSTN
LISDFLLLLCASQQWQVFSAERTEEWQRMAGVNTDRLEPLRGEPNPVPNFIHCRSYL
DMLKVAVFRYLFWLVLVVVFVTGATRISIFGLGYLLACFYLLLFGTALLQRDTRARL
VLWDCLILYNVTVIISKNMLSLLACVFVEQMQTGFCWVIQLFSLVCTVKGYYDPKEM
MDRDQDCLLPVEEAGIIWDSVCFFFLLLQRRVFLSHYYLHVRADLQATALLASRGFA
LYNAANLKSIDFHRRIEEKSLAQLKRQMERIRAKQEKHRQGRVDRSRPQDTLGPKDP
GLEPGPDSPGGSSPPRRQWWRPWLDHATVIHSGDYFLFESDSEEEEEAVPEDPRPSAQ
SAFQLAYQAWVTNAQAVLRRRQQEQEQARQEQAGQLPTGGGPSQEVEPAEGPEEA
AAGRSHVVQRVLSTAQFLWMLGQALVDELTRWLQEFTRHHGTMSDVLRAERYLLT
QELLQGGEVHRGVLDQLYTSQAEATLPGPTEAPNAPSTVSSGLGAEEPLSSMTDDMG
SPLSTGYHTRSGSEEAVTDPGEREAGASLYQGLMRTASELLLDRRLRIPELEEAELFA
EGQGRALRLLRAVYQCVAAHSELLCYFIIILNHMVTASAGSLVLPVLVFLWAMLSIPR
PSKRFWMTAIVFTEIAVVVKYLFQFGFFPWNSHVVLRRYENKPYFPPRILGLEKTDGY
IKYDLVQLMALFFHRSQLLCYGLWDHEEDSPSKEHDKSGEEEQGAEEGPGVPAATTE
DHIQVEARVGPTDGTPEPQVELRPRDTRRISLRFRRRKKEGPARKGAAAIEAEDREEE
EGEEEKEAPTGREKRPSRSGGRVRAAGRRLQGFCLSLAQGTYRPLRRFFHDILHTKYR
AATDVYALMFLADVVDFIIIIFGFWAFGKHSAATDITSSLSDDQVPEAFLVMLLIQFST
MVVDRALYLRKTVLGKLAFQVALVLAIHLWMFFILPAVTERMENQNVVAQLWYFV
KCIYFALSAYQIRCGYPTRILGNFLTKKYNHLNLFLFQGFRLVPFLVELRAVMDWVW
TDTTLSLSSWMCVEDIYANIFIIKCSRETEKKYPQPKGQKKKKIVKYGMGGLIILFLIAI
IWFPLLFMSLVRSVVGVVNQPIDVTVTLKLGGYEPLFTMSAQQPSIIPFTAQAYEELSR
QFDPQPLAMQFISQYSPEDIVTAQIEGSSGALWRISPPSRAQMKRELYNGTADITLRFT
WNFQRDLAKGGTVEYANEKHMLALAPNSTARRQLASLLEGTSDQSVVIPNLFPKYIR
APNGPEANPVKQLQPNEEADYLGVRIQLRREQGAGATGFLEWWVIELQECRTDCNL
LPMVIFSDKVSPPSLGFLAGYGIMGLYVSIVLVIGKFVRGFFSEISHSIMFEELPCVDRIL
KLCQDIFLVRETRELELEEELYAKLIFLYRSPETMIKWTREKE
(ΔL15-20 from Piezo1 AA)
SEQ ID NO: 2
LPCALL
(F2114 from Piezo1 AA)
SEQ ID NO: 3
RSVVGVVNQPIDVTVTLKLGGYEPLFTMSAQQPSIIPFTAQAYEELSRQFDPQPLAMQ
FISQYSPEDIVTAQIEGSSGALWRISPPSRAQMKRELYNGTADITLRFTWNFQRDLAKG
GTVEYANEKHMLALAPNSTARRQLASLLEGTSDQSVVIPNLFPKYIRAPNGPEANPV
KQLQPNEEADYLGVRIQLRREQGAGATGFLEWWVIELQECRTDCNLLPMVIFSDKVS
PPS
(Piezo1 nuc)
SEQ ID NO: 4
ATGGAGCCGC ACGTGCTCGG CGCGGTCCTG TACTGGCTGC TGCTGCCCTG
CGCGCTGCTG GCTGCCTGCC TGCTCCGCTT CAGCGGACTC TCGCTGGTCT
ACCTGCTCTT CCTGCTGCTG CTGCCCTGGT TCCCCGGCCC CACCCGATGC
GGCCTCCAAG GTCACACAGG CCGCCTCCTG CGGGCATTGC TGGGCCTCAG
CCTGCTCTTC CTGGTGGCCC ATCTCGCCCT CCAGATCTGC CTGCATATTG
TGCCCCGCCT GGACCAGCTC CTGGGACCCA GCTGCAGCCG CTGGGAGACC
CTCTCGCGAC ACATAGGGGT CACAAGGCTG GACCTGAAGG ACATCCCCAA
CGCCATCCGG CTGGTGGCCC CTGACCTGGG CATCTTGGTG GTCTCCTCTG
TCTGCCTCGG CATCTGCGGG CGCCTTGCAA GGAACACCCG GCAGAGCCCA
CATCCACGGG AGCTGGATGA TGATGAGAGG GATGTGGATG CCAGCCCGAC
GGCAGGGCTG CAGGAAGCAG CAACGCTGGC CCCTACACGG AGGTCACGGC
TGGCCGCTCG TTTCCGAGTC ACGGCCCACT GGCTGCTGGT GGCGGCTGGG
CGGGTCCTGG CCGTAACACT GCTTGCACTG GCAGGCATCG CCCACCCCTC
GGCCCTCTCC AGTGTCTACC TGCTGCTCTT CCTGGCCCTC TGCACCTGGT
GGGCCTGCCA CTTTCCCATC AGCACTCGGG GCTTCAGCAG ACTCTGCGTC
GCGGTGGGGT GCTTCGGCGC CGGCCATCTC ATCTGCCTCT ACTGCTACCA
GATGCCCTTG GCACAGGCTC TGCTCCCGCC TGCCGGCATC TGGGCTAGGG
TGCTGGGTCT CAAGGACTTC GTGGGTCCCA CCAACTGCTC CAGCCCCCAC
GCGCTGGTCC TCAACACCGG CCTGGACTGG CCTGTGTATG CCAGCCCCGG
CGTCCTCCTG CTGCTGTGCT ACGCCACGGC CTCTCTGCGC AAGCTCCGCG
CGTACCGCCC CTCCGGCCAG AGGAAGGAGG CGGCAAAGGG GTATGAGGCT
CGGGAGCTGG AGCTAGCAGA GCTGGACCAG TGGCCCCAGG AACGGGAGTC
TGACCAGCAC GTGGTGCCCA CAGCACCCGA CACCGAGGCT GATAACTGCA
TCGTGCACGA GCTGACCGGC CAGAGCTCCG TCCTGCGGCG GCCTGTGCGG
CCCAAGCGGG CTGAGCCCAG GGAGGCGTCT CCGCTCCACA GCCTGGGCCA
CCTCATCATG GACCAGAGCT ATGTGTGCGC GCTCATTGCC ATGATGGTAT
GGAGCATCAC CTACCACAGC TGGCTGACCT TCGTACTGCT GCTCTGGGCC
TGCCTCATCT GGACGGTGCG CAGCCGCCAC CAACTGGCCA TGCTGTGCTC
GCCCTGCATC CTGCTGTATG GGATGACGCT GTGCTGCCTA CGCTACGTGT
GGGCCATGGA CCTGCGCCCT GAGCTGCCCA CCACCCTGGG CCCCGTCAGC
CTGCGCCAGC TGGGGCTGGA GCACACCCGC TACCCCTGTC TGGACCTTGG
TGCCATGTTG CTCTACACCC TGACCTTCTG GCTCCTGCTG CGCCAGTTTG
TGAAAGAGAA GCTGCTGAAG TGGGCAGAGT CTCCAGCTGC GCTGACGGAG
GTCACCGTGG CAGACACAGA GCCCACGCGG ACGCAGACGC TGTTGCAGAG
CCTGGGGGAG CTGGTGAAGG GCGTGTACGC CAAGTACTGG ATCTATGTGT
GTGCTGGCAT GTTCATCGTG GTCAGCTTCG CCGGCCGCCT CGTGGTCTAC
AAGATTGTCT ACATGTTCCT CTTCCTGCTC TGCCTCACCC TCTTCCAGGT
CTACTACAGC CTGTGGCGGA AGCTGCTCAA GGCCTTCTGG TGGCTCGTGG
TGGCCTACAC CATGCTGGTC CTCATCGCCG TCTACACCTT CCAGTTCCAG
GACTTCCCTG CCTACTGGCG CAACCTCACT GGCTTCACCG ACGAGCAGCT
GGGGGACCTG GGCCTGGAGC AGTTCAGCGT GTCCGAGCTC TTCTCCAGCA
TCCTGGTGCC CGGCTTCTTC CTCCTGGCCT GCATCCTGCA GCTGCACTAC
TTCCACAGGC CCTTCATGCA GCTCACCGAC ATGGAGCACG TGTCCCTGCC
TGGCACGCGC CTCCCGCGCT GGGCTCACAG GCAGGATGCA GTGAGTGGGA
CCCCACTGCT GCGGGAGGAG CAGCAGGAGC ATCAGCAGCA GCAGCAGGAG
GAGGAGGAGG AGGAGGAGGA CTCCAGGGAC GAGGGGCTGG GCGTGGCCAC
TCCCCACCAG GCCACGCAGG TGCCTGAAGG GGCAGCCAAG TGGGGCCTGG
TGGCTGAGCG GCTGCTGGAG CTGGCAGCCG GCTTCTCGGA CGTCCTCTCA
CGCGTGCAGG TGTTCCTGCG GCGGCTGCTG GAGCTTCACG TTTTCAAGCT
GGTGGCCCTG TACACCGTCT GGGTGGCCCT GAAGGAGGTG TCGGTGATGA
ACCTGCTGCT GGTGGTGCTG TGGGCCTTCG CCCTGCCCTA CCCACGCTTC
CGGCCCATGG CCTCCTGCCT GTCCACCGTG TGGACCTGCG TCATCATCGT
GTGTAAGATG CTGTACCAGC TCAAGGTTGT CAACCCCCAG GAGTATTCCA
GCAACTGCAC CGAGCCCTTC CCCAACAGCA CCAACTTGCT GCCCACGGAG
ATCAGCCAGT CCCTGCTGTA CCGGGGGCCC GTGGACCCTG CCAACTGGTT
TGGGGTGCGG AAAGGGTTCC CCAACCTGGG CTACATCCAG AACCACCTGC
AAGTGCTGCT GCTGCTGGTA TTCGAGGCCA TCGTGTACCG GOGCCAGGAG
CACTACCGCC GGCAGCACCA GCTGGCCCCG CTGCCTGCCC AGGCCGTGTT
TGCCAGCGGC ACCCGCCAGC AGCTGGACCA GGATCTGCTC GGCTGCCTCA
AGTACTTCAT CAACTTCTTC TTCTACAAAT TCGGGCTGGA GATCTGCTTC
CTGATGGCCG TGAACGTGAT CGGGCAGCGC ATGAACTTTC TGGTGACCCT
GCACGGTTGC TGGCTGGTGG CCATCCTCAC CCGCAGGCAC CGCCAGGCCA
TTGCCCGCCT CTGGCCCAAC TACTGCCTCT TCCTGGCGCT GTTCCTGCTG
TACCAGTACC TGCTGTGCCT GGGGATGCCC CCGGCCCTGT GCATTGATTA
TCCCTGGCGC TGGAGCCGGG CCGTCCCCAT GAACTCCGCA CTCATCAAGT
GGCTGTACCT GCCTGATTTC TTCCGGGCCC CCAACTCCAC CAACCTCATC
AGCGACTTTC TCCTGCTGCT GTGCGCCTCC CAGCAGTGGC AGGTGTTCTC
AGCTGAGCGC ACAGAGGAGT GGCAGCGCAT GGCTGGCGTC AACACCGACC
GCCTGGAGCC GCTGCGGGGG GAGCCCAACC CCGTGCCCAA CTTTATCCAC
TGCAGGTCCT ACCTTGACAT GCTGAAGGTG GCCGTCTTCC GATACCTGTT
CTGGCTGGTG CTGGTGGTGG TGTTTGTCAC GGGGGCCACC CGCATCAGCA
TCTTCGGGCT GGGCTACCTG CTGGCCTGCT TCTACCTGCT GCTCTTCGGC
ACGGCCCTGC TGCAGAGGGA CACACGGGCC CGCCTCGTGC TGTGGGACTG
CCTCATTCTG TACAACGTCA CCGTCATCAT CTCCAAGAAC ATGCTGTCGC
TCCTGGCCTG CGTCTTCGTG GAGCAGATGC AGACCGGCTT CTGCTGGGTC
ATCCAGCTCT TCAGCCTTGT ATGCACCGTC AAGGGCTACT ATGACCCCAA
GGAGATGATG GACAGAGACC AGGACTGCCT GCTGCCTGTG GAGGAGGCTG
GCATCATCTG GGACAGCGTC TGCTTCTTCT TCCTGCTGCT GCAGCGCCGC
GTCTTCCTTA GCCATTACTA CCTGCACGTC AGGGCCGACC TCCAGGCCAC
CGCCCTGCTA GCCTCCAGGG GCTTCGCCCT CTACAACGCT GCCAACCTCA
AGAGCATTGA CTTTCACCGC AGGATAGAGG AGAAGTCCCT GGCCCAGCTG
AAAAGACAGA TGGAGCGTAT CCGTGCCAAG CAGGAGAAGC ACAGGCAGGG
CCGGGTGGAC CGCAGTCGCC CCCAGGACAC CCTGGGCCCC AAGGACCCCG
GCCTGGAGCC AGGGCCCGAC AGTCCAGGGG GCTCCTCCCC GCCACGGAGG
CAGTGGTGGC GGCCCTGGCT GGACCACGCC ACAGTCATCC ACTCCGGGGA
CTACTTCCTG TTTGAGTCCG ACAGTGAGGA AGAGGAGGAG GCTGTTCCTG
AAGACCCGAG GCCGTCGGCA CAGAGTGCCT TCCAGCTGGC GTACCAGGCA
TGGGTGACCA ACGCCCAGGC GGTGCTGAGG CGGCGGCAGC AGGAGCAGGA
GCAGGCAAGG CAGGAACAGG CAGGACAGCT ACCCACAGGA GGTGGTCCCA
GCCAGGAGGT GGAGCCAGCA GAGGGCCCCG AGGAGGCAGC GGCAGGCCGG
AGCCATGTGG TGCAGAGGGT GCTGAGCACG GCGCAGTTCC TGTGGATGCT
GGGGCAGGCG CTAGTGGATG AGCTGACACG CTGGCTGCAG GAGTTCACCC
GGCACCACGG CACCATGAGC GACGTGCTGC GGGCAGAGCG CTACCTCCTC
ACACAGGAGC TCCTGCAGGG CGGCGAAGTG CACAGGGGCG TGCTGGATCA
GCTGTACACA AGCCAGGCCG AGGCCACGCT GCCAGGCCCC ACCGAGGCCC
CCAATGCCCC AAGCACCGTG TCCAGTGGGC TGGGCGCGGA GGAGCCACTC
AGCAGCATGA CAGACGACAT GGGCAGCCCC CTGAGCACCG GCTACCACAC
GCGCAGTGGC AGTGAGGAGG CAGTCACCGA CCCCGGGGAG CGTGAGGCTG
GTGCCTCTCT GTACCAGGGA CTGATGCGGA CGGCCAGCGA GCTGCTCCTG
GACAGGCGCC TGCGCATCCC AGAGCTGGAG GAGGCAGAGC TGTTTGCGGA
GGGGCAGGGC CGGGCGCTGC GGCTGCTGCG GGCCGTGTAC CAGTGTGTGG
CCGCCCACTC GGAGCTGCTC TGCTACTTCA TCATCATCCT CAACCACATG
GTCACGGCCT CCGCCGGCTC GCTGGTGCTG CCCGTGCTCG TCTTCCTGTG
GGCCATGCTG TCGATCCCGA GGCCCAGCAA GCGCTTCTGG ATGACGGCCA
TCGTCTTCAC CGAGATCGCG GTGGTCGTCA AGTACCTGTT CCAGTTTGGG
TTCTTCCCCT GGAACAGCCA CGTGGTGCTG CGGCGCTACG AGAACAAGCC
CTACTTCCCG CCCCGCATCC TGGGCCTGGA GAAGACTGAC GGCTACATCA
AGTACGACCT GGTGCAGCTC ATGGCCCTTT TCTTCCACCG CTCCCAGCTG
CTGTGCTATG GCCTCTGGGA CCATGAGGAG GACTCACCAT CCAAGGAGCA
TGACAAGAGC GGCGAGGAGG AGCAGGGAGC CGAGGAGGGG CCAGGGGTGC
CTGCGGCCAC CACCGAAGAC CACATTCAGG TGGAAGCCAG GGTCGGACCC
ACGGACGGGA CCCCAGAACC CCAAGTGGAG CTCAGGCCCC GTGATACGAG
GCGCATCAGT CTACGTTTTA GAAGAAGGAA GAAGGAGGGC CCAGCACGGA
AAGGAGCGGC AGCCATCGAA GCTGAGGACA GGGAGGAAGA AGAGGGGGAG
GAAGAGAAAG AGGCCCCCAC GGGGAGAGAG AAGAGGCCAA GCCGCTCTGG
AGGAAGAGTA AGGGCGGCCG GGCGGCGGCT GCAGGGCTTC TGCCTGTCCC
TGGCCCAGGG CACATATCGG CCGCTACGGC GCTTCTTCCA CGACATCCTG
CACACCAAGT ACCGCGCAGC CACCGACGTC TATGCCCTCA TGTTCCTGGC
TGATGTTGTC GACTTCATCA TCATCATTTT TGGCTTCTGG GCCTTTGGGA
AGCACTCGGC GGCCACAGAC ATCACGTCCT CCCTATCAGA CGACCAGGTA
CCCGAGGCTT TCCTGGTCAT GCTGCTGATC CAGTTCAGTA CCATGGTGGT
TGACCGCGCC CTCTACCTGC GCAAGACCGT GCTGGGCAAG CTGGCCTTCC
AGGTGGCGCT GGTGCTGGCC ATCCACCTAT GGATGTTCTT CATCCTGCCC
GCCGTCACTG AGAGGATGTT CAACCAGAAT GTGGTGGCCC AGCTCTGGTA
CTTCGTGAAG TGCATCTACT TCGCCCTGTC CGCCTACCAG ATCCGCTGCG
GCTACCCCAC CCGCATCCTC GGCAACTTCC TCACCAAGAA GTACAATCAT
CTCAACCTCT TCCTCTTCCA GGGGTTCCGG CTGGTGCCGT TCCTGGTGGA
GCTGCGGGCA GTGATGGACT GGGTGTGGAC GGACACCACG CTGTCCCTGT
CCAGCTGGAT GTGTGTGGAG GACATCTATG CCAACATCTT CATCATCAAA
TGCAGCCGAG AGACAGAGAA GAAATACCCG CAGCCCAAAG GGCAGAAGAA
GAAGAAGATC GTCAAGTACG GCATGGGTGG CCTCATCATC CTCTTCCTCA
TCGCCATCAT CTGGTTCCCA CTGCTCTTCA TGTCGCTGGT GCGCTCCGTG
GTTGGGGTTG TCAACCAGCC CATCGATGTC ACCGTCACCC TGAAGCTGGG
CGGCTATGAG CCGCTGTTCA CCATGAGCGC CCAGCAGCCG TCCATCATCC
CCTTCACGGC CCAGGCCTAT GAGGAGCTGT CCCGGCAGTT TGACCCCCAG
CCGCTGGCCA TGCAGTTCAT CAGCCAGTAC AGCCCTGAGG ACATCGTCAC
GGCGCAGATT GAGGGCAGCT CCGGGGCGCT GTGGCGCATC AGTCCCCCCA
GCCGTGCCCA GATGAAGCGG GAGCTCTACA ACGGCACGGC CGACATCACC
CTGCGCTTCA CCTGGAACTT CCAGAGGGAC CTGGCGAAGG GAGGCACTGT
GGAGTATGCC AACGAGAAGC ACATGCTGGC CCTGGCCCCC AACAGCACTG
CACGGCGGCA GCTGGCCAGC CTGCTCGAGG GCACCTCGGA CCAGTCTGTG
GTCATCCCTA ATCTCTTCCC CAAGTACATC CGTGCCCCCA ACGGGCCCGA
AGCCAACCCT GTGAAGCAGC TGCAGCCCAA TGAGGAGGCC GACTACCTCG
GCGTGCGTAT CCAGCTGCGG AGGGAGCAGG GTGCGGGGGC CACCGGCTTC
CTCGAATGGT GGGTCATCGA GCTGCAGGAG TGCCGGACCG ACTGCAACCT
GCTGCCCATG GTCATTTTCA GTGACAAGGT CAGCCCACCG AGCCTCGGCT
TCCTGGCTGG CTACGGCATC ATGGGGCTGT ACGTGTCCAT CGTGCTGGTC
ATCGGCAAGT TCGTGCGCGG ATTCTTCAGC GAGATCTCGC ACTCCATTAT
GTTCGAGGAG CTGCCGTGCG TGGACCGCAT CCTCAAGCTC TGCCAGGACA
TCTTCCTGGT GCGGGAGACT CGGGAGCTGG AGCTGGAGGA GGAGTTGTAC
GCCAAGCTCA TCTTCCTCTA CCGCTCACCO GAGACCATGA TCAAGTGGAC
TCGTGAGAAG GAGTAG
(ΔL15-20 from Piezo1 Nuc)
SEQ ID NO: 5
CTGCCCTGCGCGCTGCTG
(F2114 from Piezo1 Nuc)
SEQ ID NO: 6
CAGCCCATCGATGTCACCGTCACCCTGAAGCTGGGCGGCTATGAGCCGCTGTTCA
CCATGAGCGCCCAGCAGCCGTCCATCATCCCCTTCACGGCCCAGGCCTATGAGGA
GCTGTCCCGGCAGTTTGACCCCCAGCCGCTGGCCATGCAGTTCATCAGCCAGTAC
AGCCCTGAGGACATCGTCACGGCGCAGATTGAGGGCAGCTCCGGGGCGCTGTGG
CGCATCAGTCCCCCCAGC
(P2A AA)
SEQ ID NO: 7
ATNFSLLKQAGDVEENPGP
(Bicistronic vector Nuc, P2A can be linked to nucleic acid sequence
encoding CR) Key: CMV, Piezo1, P2A
SEQ ID NO: 8
CGTTACATAACTTACGGTAAATGGCCCGCCTGGCTGACCGCCCAACGACCCC
CGCCCATTGACGTCAATAATGACGTATGTTCCCATAGTAACGCCAATAGGG
ACTTTCCATTGACGTCAATGGGTGGAGTATTTACGGTAAACTGCCCACTTG
GCAGTACATCAAGTGTATCATATGCCAAGTACGCCCCCTATTGACGTCAAT
GACGGTAAATGGCCCGCCTGGCATTATGCCCAGTACATGACCTTATGGGAC
TTTCCTACTTGGCAGTACATCTACGTATTAGTCATCGCTATTACCATGGTGA
TGCGGTTTTGGCAGTACATCAATGGGCGTGGATAGCGGTTTGACTCACGG
GGATTTCCAAGTCTCCACCCCATTGACGTCAATGGGAGTTTGTTTTGGCAC
CAAAATCAACGGGACTTTCCAAAATGTCGTAACAACTCCGCCCCATTGACG
CAAATGGGCGGTAGGCGTGTACGGTGGGAGGTCTATATAAGCAGAGCT
ATGGAGCCGC ACGTGCTCGG CGCGGTCCTG TACTGGCTGC TGCTGCCCTG
CGCGCTGCTG GCTGCCTGCC TGCTCCGCTT CAGCGGACTC TCGCTGGTCT
ACCTGCTCTT CCTGCTGCTG CTGCCCTGGT TCCCCGGCCC CACCCGATGC
GGCCTCCAAG GTCACACAGG CCGCCTCCTG CGGGCATTGC TGGGCCTCAG
CCTGCTCTTC CTGGTGGCCC ATCTCGCCCT CCAGATCTGC CTGCATATTG
TGCCCCGCCT GGACCAGCTC CTGGGACCCA GCTGCAGCCG CTGGGAGACC
CTCTCGCGAC ACATAGGGGT CACAAGGCTG GACCTGAAGG ACATCCCCAA
CGCCATCCGG CTGGTGGCCC CTGACCTGGG CATCTTGGTG GTCTCCTCTG
TCTGCCTCGG CATCTGCGGG CGCCTTGCAA GGAACACCCG GCAGAGCCCA
CATCCACGGG AGCTGGATGA TGATGAGAGG GATGTGGATG CCAGCCCGAC
GGCAGGGCTG CAGGAAGCAG CAACGCTGGC CCCTACACGG AGGTCACGGC
TGGCCGCTCG TTTCCGAGTC ACGGCCCACT GGCTGCTGGT GGCGGCTGGG
CGGGTCCTGG CCGTAACACT GCTTGCACTG GCAGGCATCG CCCACCCCTC
GGCCCTCTCC AGTGTCTACC TGCTGCTCTT CCTGGCCCTC TGCACCTGGT
GGGCCTGCCA CTTTCCCATC AGCACTCGGG GCTTCAGCAG ACTCTGCGTC
GCGGTGGGGT GCTTCGGCGC CGGCCATCTC ATCTGCCTCT ACTGCTACCA
GATGCCCTTG GCACAGGCTC TGCTCCCGCC TGCCGGCATC TGGGCTAGGG
TGCTGGGTCT CAAGGACTTC GTGGGTCCCA CCAACTGCTC CAGCCCCCAC
GCGCTGGTCC TCAACACCGG CCTGGACTGG CCTGTGTATG CCAGCCCCGG
CGTCCTCCTG CTGCTGTGCT ACGCCACGGC CTCTCTGCGC AAGCTCCGCG
CGTACCGCCC CTCCGGCCAG AGGAAGGAGG CGGCAAAGGG GTATGAGGCT
CGGGAGCTGG AGCTAGCAGA GCTGGACCAG TGGCCCCAGG AACGGGAGTC
TGACCAGCAC GTGGTGCCCA CAGCACCCGA CACCGAGGCT GATAACTGCA
TCGTGCACGA GCTGACCGGC CAGAGCTCCG TCCTGCGGCG GCCTGTGCGG
CCCAAGCGGG CTGAGCCCAG GGAGGCGTCT CCGCTCCACA GCCTGGGCCA
CCTCATCATG GACCAGAGCT ATGTGTGCGC GCTCATTGCC ATGATGGTAT
GGAGCATCAC CTACCACAGC TGGCTGACCT TCGTACTGCT GCTCTGGGCC
TGCCTCATCT GGACGGTGCG CAGCCGCCAC CAACTGGCCA TGCTGTGCTC
GCCCTGCATC CTGCTGTATG GGATGACGCT GTGCTGCCTA CGCTACGTGT
GGGCCATGGA CCTGCGCCCT GAGCTGCCCA CCACCCTGGG CCCCGTCAGC
CTGCGCCAGC TGGGGCTGGA GCACACCCGC TACCCCTGTC TGGACCTTGG
TGCCATGTTG CTCTACACCC TGACCTTCTG GCTCCTGCTG CGCCAGTTTG
TGAAAGAGAA GCTGCTGAAG TGGGCAGAGT CTCCAGCTGC GCTGACGGAG
GTCACCGTGG CAGACACAGA GCCCACGCGG ACGCAGACGC TGTTGCAGAG
CCTGGGGGAG CTGGTGAAGG GCGTGTACGC CAAGTACTGG ATCTATGTGT
GTGCTGGCAT GTTCATCGTG GTCAGCTTCG CCGGCCGCCT CGTGGTCTAC
AAGATTGTCT ACATGTTCCT CTTCCTGCTC TGCCTCACCC TCTTCCAGGT
CTACTACAGC CTGTGGCGGA AGCTGCTCAA GGCCTTCTGG TGGCTCGTGG
TGGCCTACAC CATGCTGGTC CTCATCGCCG TCTACACCTT CCAGTTCCAG
GACTTCCCTG CCTACTGGCG CAACCTCACT GGCTTCACCG ACGAGCAGCT
GGGGGACCTG GGCCTGGAGC AGTTCAGCGT GTCCGAGCTC TTCTCCAGCA
TCCTGGTGCC CGGCTTCTTC CTCCTGGCCT GCATCCTGCA GCTGCACTAC
TTCCACAGGC CCTTCATGCA GCTCACCGAC ATGGAGCACG TGTCCCTGCC
TGGCACGCGC CTCCCGCGCT GGGCTCACAG GCAGGATGCA GTGAGTGGGA
CCCCACTGCT GCGGGAGGAG CAGCAGGAGC ATCAGCAGCA GCAGCAGGAG
GAGGAGGAGG AGGAGGAGGA CTCCAGGGAC GAGGGGCTGG GCGTGGCCAC
TCCCCACCAG GCCACGCAGG TGCCTGAAGG GGCAGCCAAG TGGGGCCTGG
TGGCTGAGCG GCTGCTGGAG CTGGCAGCCG GCTTCTCGGA CGTCCTCTCA
CGCGTGCAGG TGTTCCTGCG GCGGCTGCTG GAGCTTCACG TTTTCAAGCT
GGTGGCCCTG TACACCGTCT GGGTGGCCCT GAAGGAGGTG TCGGTGATGA
ACCTGCTGCT GGTGGTGCTG TGGGCCTTCG CCCTGCCCTA CCCACGCTTC
CGGCCCATGG CCTCCTGCCT GTCCACCGTG TGGACCTGCG TCATCATCGT
GTGTAAGATG CTGTACCAGC TCAAGGTTGT CAACCCCCAG GAGTATTCCA
GCAACTGCAC CGAGCCCTTC CCCAACAGCA CCAACTTGCT GCCCACGGAG
ATCAGCCAGT CCCTGCTGTA CCGGGGGCCC GTGGACCCTG CCAACTGGTT
TGGGGTGCGG AAAGGGTTCC CCAACCTGGG CTACATCCAG AACCACCTGC
AAGTGCTGCT GCTGCTGGTA TTCGAGGCCA TCGTGTACCG GCGCCAGGAG
CACTACCGCC GGCAGCACCA GCTGGCCCCG CTGCCTGCCC AGGCCGTGTT
TGCCAGCGGC ACCCGCCAGC AGCTGGACCA GGATCTGCTC GGCTGCCTCA
AGTACTTCAT CAACTTCTTC TTCTACAAAT TCGGGCTGGA GATCTGCTTC
CTGATGGCCG TGAACGTGAT CGGGCAGCGC ATGAACTTTC TGGTGACCCT
GCACGGTTGC TGGCTGGTGG CCATCCTCAC CCGCAGGCAC CGCCAGGCCA
TTGCCCGCCT CTGGCCCAAC TACTGCCTCT TCCTGGCGCT GTTCCTGCTG
TACCAGTACC TGCTGTGCCT GGGGATGCCC CCGGCCCTGT GCATTGATTA
TCCCTGGCGC TGGAGCCGGG CCGTCCCCAT GAACTCCGCA CTCATCAAGT
GGCTGTACCT GCCTGATTTC TTCCGGGCCC CCAACTCCAC CAACCTCATC
AGCGACTTTC TCCTGCTGCT GTGCGCCTCC CAGCAGTGGC AGGTGTTCTC
AGCTGAGCGC ACAGAGGAGT GGCAGCGCAT GGCTGGCGTC AACACCGACC
GCCTGGAGCC GCTGCGGGGG GAGCCCAACC CCGTGCCCAA CTTTATCCAC
TGCAGGTCCT ACCTTGACAT GCTGAAGGTG GCCGTCTTCC GATACCTGTT
CTGGCTGGTG CTGGTGGTGG TGTTTGTCAC GGGGGCCACC CGCATCAGCA
TCTTCGGGCT GGGCTACCTG CTGGCCTGCT TCTACCTGCT GCTCTTCGGC
ACGGCCCTGC TGCAGAGGGA CACACGGGCC CGCCTCGTGC TGTGGGACTG
CCTCATTCTG TACAACGTCA CCGTCATCAT CTCCAAGAAC ATGCTGTCGC
TCCTGGCCTG CGTCTTCGTG GAGCAGATGC AGACCGGCTT CTGCTGGGTC
ATCCAGCTCT TCAGCCTTGT ATGCACCGTC AAGGGCTACT ATGACCCCAA
GGAGATGATG GACAGAGACC AGGACTGCCT GCTGCCTGTG GAGGAGGCTG
GCATCATCTG GGACAGCGTC TGCTTCTTCT TCCTGCTGCT GCAGCGCCGC
GTCTTCCTTA GCCATTACTA CCTGCACGTC AGGGCCGACC TCCAGGCCAC
CGCCCTGCTA GCCTCCAGGG GCTTCGCCCT CTACAACGCT GCCAACCTCA
AGAGCATTGA CTTTCACCGC AGGATAGAGG AGAAGTCCCT GGCCCAGCTG
AAAAGACAGA TGGAGCGTAT CCGTGCCAAG CAGGAGAAGC ACAGGCAGGG
CCGGGTGGAC CGCAGTCGCC CCCAGGACAC CCTGGGCCCC AAGGACCCCG
GCCTGGAGCC AGGGCCCGAC AGTCCAGGGG GCTCCTCCCC GCCACGGAGG
CAGTGGTGGC GGCCCTGGCT GGACCACGCC ACAGTCATCC ACTCCGGGGA
CTACTTCCTG TTTGAGTCCG ACAGTGAGGA AGAGGAGGAG GCTGTTCCTG
AAGACCCGAG GCCGTCGGCA CAGAGTGCCT TCCAGCTGGC GTACCAGGCA
TGGGTGACCA ACGCCCAGGC GGTGCTGAGG CGGCGGCAGC AGGAGCAGGA
GCAGGCAAGG CAGGAACAGG CAGGACAGCT ACCCACAGGA GGTGGTCCCA
GCCAGGAGGT GGAGCCAGCA GAGGGCCCCG AGGAGGCAGC GGCAGGCCGG
AGCCATGTGG TGCAGAGGGT GCTGAGCACG GCGCAGTTCC TGTGGATGCT
GGGGCAGGCG CTAGTGGATG AGCTGACACG CTGGCTGCAG GAGTTCACCC
GGCACCACGG CACCATGAGC GACGTGCTGC GGGCAGAGCG CTACCTCCTC
ACACAGGAGC TCCTGCAGGG CGGCGAAGTG CACAGGGGCG TGCTGGATCA
GCTGTACACA AGCCAGGCCG AGGCCACGCT GCCAGGCCCC ACCGAGGCCC
CCAATGCCCC AAGCACCGTG TCCAGTGGGC TGGGCGCGGA GGAGCCACTC
AGCAGCATGA CAGACGACAT GGGCAGCCCC CTGAGCACCG GCTACCACAC
GCGCAGTGGC AGTGAGGAGG CAGTCACCGA CCCCGGGGAG CGTGAGGCTG
GTGCCTCTCT GTACCAGGGA CTGATGCGGA CGGCCAGCGA GCTGCTCCTG
GACAGGCGCC TGCGCATCCC AGAGCTGGAG GAGGCAGAGC TGTTTGCGGA
GGGGCAGGGC CGGGCGCTGC GGCTGCTGCG GGCCGTGTAC CAGTGTGTGG
CCGCCCACTC GGAGCTGCTC TGCTACTTCA TCATCATCCT CAACCACATG
GTCACGGCCT CCGCCGGCTC GCTGGTGCTG CCCGTGCTCG TCTTCCTGTG
GGCCATGCTG TCGATCCCGA GGCCCAGCAA GCGCTTCTGG ATGACGGCCA
TCGTCTTCAC CGAGATCGCG GTGGTCGTCA AGTACCTGTT CCAGTTTGGG
TTCTTCCCCT GGAACAGCCA CGTGGTGCTG CGGCGCTACG AGAACAAGCC
CTACTTCCCG CCCCGCATCC TGGGCCTGGA GAAGACTGAC GGCTACATCA
AGTACGACCT GGTGCAGCTC ATGGCCCTTT TCTTCCACCG CTCCCAGCTG
CTGTGCTATG GCCTCTGGGA CCATGAGGAG GACTCACCAT CCAAGGAGCA
TGACAAGAGC GGCGAGGAGG AGCAGGGAGC CGAGGAGGGG CCAGGGGTGC
CTGCGGCCAC CACCGAAGAC CACATTCAGG TGGAAGCCAG GGTCGGACCC
ACGGACGGGA CCCCAGAACC CCAAGTGGAG CTCAGGCCCC GTGATACGAG
GCGCATCAGT CTACGTTTTA GAAGAAGGAA GAAGGAGGGC CCAGCACGGA
AAGGAGCGGC AGCCATCGAA GCTGAGGACA GGGAGGAAGA AGAGGGGGAG
GAAGAGAAAG AGGCCCCCAC GGGGAGAGAG AAGAGGCCAA GCCGCTCTGG
AGGAAGAGTA AGGGCGGCCG GGCGGCGGCT GCAGGGCTTC TGCCTGTCCC
TGGCCCAGGG CACATATCGG CCGCTACGGC GCTTCTTCCA CGACATCCTG
CACACCAAGT ACCGCGCAGC CACCGACGTC TATGCCCTCA TGTTCCTGGC
TGATGTTGTC GACTTCATCA TCATCATTTT TGGCTTCTGG GCCTTTGGGA
AGCACTCGGC GGCCACAGAC ATCACGTCCT CCCTATCAGA CGACCAGGTA
CCCGAGGCTT TCCTGGTCAT GCTGCTGATC CAGTTCAGTA CCATGGTGGT
TGACCGCGCC CTCTACCTGC GCAAGACCGT GCTGGGCAAG CTGGCCTTCC
AGGTGGCGCT GGTGCTGGCC ATCCACCTAT GGATGTTCTT CATCCTGCCC
GCCGTCACTG AGAGGATGTT CAACCAGAAT GTGGTGGCCC AGCTCTGGTA
CTTCGTGAAG TGCATCTACT TCGCCCTGTC CGCCTACCAG ATCCGCTGCG
GCTACCCCAC CCGCATCCTC GGCAACTTCC TCACCAAGAA GTACAATCAT
CTCAACCTCT TCCTCTTCCA GGGGTTCCGG CTGGTGCCGT TCCTGGTGGA
GCTGCGGGCA GTGATGGACT GGGTGTGGAC GGACACCACG CTGTCCCTGT
CCAGCTGGAT GTGTGTGGAG GACATCTATG CCAACATCTT CATCATCAAA
TGCAGCCGAG AGACAGAGAA GAAATACCCG CAGCCCAAAG GGCAGAAGAA
GAAGAAGATC GTCAAGTACG GCATGGGTGG CCTCATCATC CTCTTCCTCA
TCGCCATCAT CTGGTTCCCA CTGCTCTTCA TGTCGCTGGT GCGCTCCGTG
GTTGGGGTTG TCAACCAGCC CATCGATGTC ACCGTCACCC TGAAGCTGGG
CGGCTATGAG CCGCTGTTCA CCATGAGCGC CCAGCAGCCG TCCATCATCC
CCTTCACGGC CCAGGCCTAT GAGGAGCTGT CCCGGCAGTT TGACCCCCAG
CCGCTGGCCA TGCAGTTCAT CAGCCAGTAC AGCCCTGAGG ACATCGTCAC
GGCGCAGATT GAGGGCAGCT CCGGGGCGCT GTGGCGCATC AGTCCCCCCA
GCCGTGCCCA GATGAAGCGG GAGCTCTACA ACGGCACGGC CGACATCACC
CTGCGCTTCA CCTGGAACTT CCAGAGGGAC CTGGCGAAGG GAGGCACTGT
GGAGTATGCC AACGAGAAGC ACATGCTGGC CCTGGCCCCC AACAGCACTG
CACGGCGGCA GCTGGCCAGC CTGCTCGAGG GCACCTCGGA CCAGTCTGTG
GTCATCCCTA ATCTCTTCCC CAAGTACATC CGTGCCCCCA ACGGGCCCGA
AGCCAACCCT GTGAAGCAGC TGCAGCCCAA TGAGGAGGCC GACTACCTCG
GCGTGCGTAT CCAGCTGCGG AGGGAGCAGG GTGCGGGGGC CACCGGCTTC
CTCGAATGGT GGGTCATCGA GCTGCAGGAG TGCCGGACCG ACTGCAACCT
GCTGCCCATG GTCATTTTCA GTGACAAGGT CAGCCCACCG AGCCTCGGCT
TCCTGGCTGG CTACGGCATC ATGGGGCTGT ACGTGTCCAT CGTGCTGGTC
ATCGGCAAGT TCGTGCGCGG ATTCTTCAGC GAGATCTCGC ACTCCATTAT
GTTCGAGGAG CTGCCGTGCG TGGACCGCAT CCTCAAGCTC TGCCAGGACA
TCTTCCTGGT GCGGGAGACT CGGGAGCTGG AGCTGGAGGA GGAGTTGTAC
GCCAAGCTCA TCTTCCTCTA CCGCTCACCG GAGACCATGA TCAAGTGGAC
TCGTGAGAAG GAGTAGGGCAGCGGCGCCACAAACTTCTCTCTGCTAAAGCAA
GCAGGTGATGTTGAAGAAAACCCCGGGCCT

What is claimed is:

1. A method of producing a Chimeric Antigen Receptor (CAR)-expressing immune cell comprising introducing a first nucleic acid sequence encoding a CAR and a second nucleic acid sequence encoding a mechanosensitive ligand to an immune cell.

2. The method of claim 1, wherein the first nucleic acid sequence encodes a CAR that binds CD19, BCMA, CD123, or Mesothelin.

3. The method of claim 2, wherein the first nucleic acid sequence encodes a CAR molecule comprising a CD8 leader sequence, a CD19-scFv, a CD28-hinge region, a transmembrane domain derived from CD28 and/or 4-1BB, and a cytoplasmic signaling domain of CD3ζ.

4. The method of claim 1, wherein the second nucleic acid sequence encodes a mechanosensitive ligand selected from the group consisting of Piezo1, a fragment of Piezo1 comprising the ΔL15-20 extracellular loop, a fragment of Piezo1 comprising the F2114 residue, fragments thereof, and variants thereof.

5. The method of claim 1, wherein the second nucleic acid sequence encodes an amino acid sequence comprising SEQ ID NO:1, SEQ ID NO: 2, SEQ ID NO:3, or a combination thereof.

6. The method of claim 1, wherein the second nucleic acid sequence comprises SEQ ID NO:4, SEQ ID NO:5, SEQ ID NO:6, or a combination thereof.

7. The method of claim 1, wherein the method comprises contacting the immune cell with a first nucleic acid molecule comprising the first sequence, and a second nucleic acid molecule comprising the second sequence.

8. The method of claim 1, wherein the method comprises contacting the immune cell with a nucleic acid molecule comprising the first and second nucleic acid sequence.

9. An immune cell genetically engineered to express:
a) a Chimeric Antigen Receptor (CAR); and
b) a mechanosensitive ligand.

10. The immune cell of claim 9, wherein the CAR binds to an antigen selected from the group consisting of CD19, BCMA, CD123, or Mesothelin.

11. The immune cell of claim 9, wherein the CAR comprises a CD8 leader sequence, a CD19-scFv, a CD28-hinge region, a transmembrane domain derived from CD28 and/or 4-1BB, and a cytoplasmic signaling domain of CD3ζ.

12. The immune cell of claim 9, wherein the mechanosensitive ligand is selected from the group consisting of Piezo1, a fragment of Piezo1 comprising the ΔL15-20 extracellular loop, a fragment of Piezo1 comprising the F2114 residue, fragments thereof, and variants thereof.

13. The immune cell of claim 9, wherein the mechanosensitive ligand comprises the amino acid sequence of SEQ ID NO:1, SEQ ID NO: 2, SEQ ID NO:3, or a combination thereof.

14. A composition comprising at least one nucleic acid molecule, wherein the composition comprises:
a) a first nucleic acid sequence encoding a CAR; and
b) a second nucleic acid sequence encoding a mechanosensitive ligand.

15. The composition of claim 14, wherein the composition comprises a nucleic acid molecule comprising the first nucleic acid sequence and the second nucleic acid sequence.

16. The composition of claim 14, wherein the composition comprises a first nucleic acid molecule and a second nucleic acid molecule, wherein the first nucleic acid molecule comprises a nucleic acid sequence encoding a CAR, and the second nucleic acid molecule comprises a nucleic acid sequence encoding a mechanosensitive ligand.

17. The composition of claim 14, wherein the first nucleic acid sequence encodes a CAR that binds an antigen selected from the group consisting of CD19, BCMA, CD123, and Mesothelin.

18. The composition of claim 14, wherein the first nucleic acid sequence encodes a CAR molecule comprising a CD8 leader sequence, a CD19-scFv, a CD28-hinge region, a transmembrane domain derived from CD28 and/or 4-1BB, and a cytoplasmic signaling domain of CD3ζ.

19. The composition of claim 14, wherein the second nucleic acid sequence encodes a mechanosensitive ligand selected from the group consisting of Piezo1, a fragment of Piezo1 comprising the ΔL15-20 extracellular loop, a fragment of Piezo1 comprising the F2114 residue, fragments thereof, and variants thereof.

20. The composition of claim 14, wherein the second nucleic acid sequence encodes an amino acid sequence of SEQ ID NO:1, SEQ ID NO: 2, SEQ ID NO:3, or a combination thereof.

21. The composition of claim 14, wherein the second nucleic acid sequence comprises SEQ ID NO:4, SEQ ID NO:5, SEQ ID NO:6, or a combination thereof.

22. The composition of claim 15, wherein the nucleic acid molecule encodes a fusion protein comprising a CAR and a mechanosensitive ligand linked by a self-cleaving peptide.

23. The composition of claim 22, wherein the nucleic acid molecule comprises a nucleic acid sequence of SEQ ID NO:8 linked to a nucleic acid sequence encoding a CAR.
